(19) United States
(12) Patent Application Publication
(10) Pub. No.: US 2022/0046985 A1
(43) Pub. Date: Feb. 17, 2022

(54) APPARATUS AND METHODS FOR SERIAL CONFIGURATIONS OF MULTI-CHAMBER VAPORIZATION DEVICES

(71) Applicant: HEXO OPERATIONS INC., Ottawa (CA)

(72) Inventors: Patrick WOODS, Ottawa (CA); Max ALSAYAR, Ottawa (CA); Guy DEGRACE, Ottawa (CA)

(21) Appl. No.: 17/416,720

(22) PCT Filed: Dec. 20, 2019
(86) PCT No.: PCT/CA2019/051880

§ 371 Date: Jun. 21, 2021

Related U.S. Application Data
(60) Provisional application No. 62/783,369, filed on Dec. 21, 2018.
(60) Provisional application No. 62/792,599, filed on Jan. 15, 2019.
(60) Provisional application No. 62/938,996, filed on Nov. 22, 2019.

(51) Int. Cl. A24F 40/30; A24F 40/485; A24F 40/44; A24F 40/60; A24F 40/46

(52) U.S. Cl. CPC A24F 40/30; A24F 40/485; A24F 40/46; A24F 40/60; A24F 40/44

(57) ABSTRACT

An apparatus includes a first chamber to store a first vaporization substance and an atomizer, in fluid communication with the first chamber, to generate vapor from the first vaporization substance by heating the first vaporization substance. Some embodiments include a channel, in fluid communication with the atomizer, a second chamber to store a second vaporization substance, and a feeder, in fluid communication with the channel and the second chamber, to feed the second vaporization substance from the second chamber to the channel. A heater could be provided in fluid communication with the atomizer, to heat the vapor from the atomizer. The vapor that is heated by the heater could vaporize the second vaporization substance.

### CROSS-REFERENCE TO RELATED APPLICATIONS

This application is related to, and claims priority to, U.S. Provisional Patent Application No. 62/783,369, entitled “APPARATUS AND METHODS FOR SERIAL CONFIGURATIONS OF MULTI-CHAMBER VAPORIZATION DEVICES”, and filed on Dec. 21, 2018; U.S. Provisional Patent Application No. 62/792,599, entitled “VAPORIZATION DEVICE WITH RESIDUE PREVENTION OR REDUCTION”, and filed on Jan. 15, 2019; and U.S. Provisional Patent Application No. 62/938,996, entitled “VAPORIZATION DEVICE WITH VAPOR COOLING”, and filed on Nov. 22, 2019, the entire contents of each of which are incorporated by reference herein.

### FIELD

This application relates generally to vaporization devices, and in particular to serial configurations for multi-chamber vaporization devices.

### BACKGROUND

A vaporization device is used to vaporize substances for inhalation. These substances are referred to herein as vaporization substances, and could include, for example, cannabis products, tobacco products, herbs, and/or flavorants. In some cases, substances in cannabis, tobacco, or other plants or materials extracted to generate concentrates are used as vaporization substances. These substances could include cannabinoids from cannabis, and nicotine from tobacco. In other cases, synthetic substances are artificially manufactured. Terpenes are common flavorant vaporization substances, and could be generated from natural essential oils or artificially.

Vaporization substances could be in the form of loose leaf in the case of cannabis, tobacco, and herbs, for example, or in the form of concentrates or derivative products such as liquids, waxes, or gels, for example. Vaporization substances, whether intended for flavor or some other effect, could be mixed with other compounds such as propylene glycol, glycerin, medium chain triglyceride (MCT) oil and/or water to adjust the viscosity of a final vaporization substance.

In a vaporization device, the vaporization substance is heated to the vaporization temperature of one or more constituents of the vaporization substance. This produces a vapor, which may also be referred to as an aerosol. The vapor is then inhaled by a user through a channel that is provided in the vaporization device, and often through a hose or pipe that is part of or attached to the vaporization device.

### SUMMARY

According to an aspect of the present disclosure, an apparatus includes a first chamber to store a first vaporization substance; an atomizer, in fluid communication with the first chamber, to generate vapor from the first vaporization substance by heating the first vaporization substance; a channel, in fluid communication with the atomizer; a second chamber to store a second vaporization substance; a feeder, in fluid communication with the channel and the second chamber, to feed the second vaporization substance from the second chamber to the channel.

The feeder is downstream from the atomizer in some embodiments.

The second vaporization substance could be vaporized, for example, by heat from the vapor.

The feeder could be or include an unheated atomizer.

An apparatus could include a mouthpiece in fluid communication with the channel.

Regarding the feeder, at least a portion of the feeder could be inside the channel, or in a separate channel that is in fluid communication with the channel. The feeder could be or include a regulator to control movement of the second vaporization substance from the second chamber to the channel. An apparatus could also include a user input device to control the regulator to permit or inhibit the movement of the second vaporization substance from the second chamber to the channel.

The regulator could be or include any one or more of: a wick, a valve, a pump, a spray nozzle to spray the second vaporization substance, and a mechanical feed structure such as a screw conveyor.

Another example of a feeder is a holder to hold the second vaporization substance in the channel or the separate channel. An apparatus could include a user input device to control exposure of the holder to the channel or the separate channel. The holder could be or include, for example, any one or more of: a wick, an absorbent material, and an air permeable material.

A vapor regulator to control a flow of the vapor from the atomizer could be provided in an apparatus.

In some embodiments, the first chamber and/or the second chamber includes an engagement structure to engage with a complementary engagement structure of the apparatus.

The first chamber could include an indicator of the first vaporization substance and/or the second chamber could include an indicator of the second vaporization substance.

The first vaporization substance could be or include at least one of: a dry substance, a liquid, a gel and a wax. In some embodiments, the first vaporization substance includes an active substance.

The second vaporization substance could similarly be or include at least one of: a dry substance, a liquid, a gel and a wax. In some embodiments, the second vaporization substance includes a flavorant. The flavorant could be any one or more of: a terpene, an essential oil, and a volatile plant extract, for example.

Multiple second chambers could be provided, in which case the second chamber is one of a plurality of chambers in fluid communication with respective feeders that are in fluid communication with the channel.

A heater could be provided, in fluid communication with the atomizer, to heat the vapor from the atomizer to vaporize the second vaporization substance.

According to another aspect of the present disclosure, an apparatus includes a first chamber to store a first vaporization substance; an atomizer, in fluid communication with the first chamber, to generate vapor from the first vaporization substance by heating the first vaporization substance; a heater, in fluid communication with the atomizer, to heat the vapor from the atomizer; and a second chamber, in fluid communication with the heater, to store a second vaporization substance for vaporization by the vapor that is heated by the heater.

Such an apparatus could also include an channel in fluid communication with the atomizer and the second chamber.

A mouthpiece could be in fluid communication with the channel.

In some embodiments, at least a portion of the heater is inside the channel.

A regulator could be provided to control movement of the second vaporization substance from the second chamber to the channel. A user input device could be provided to control the regulator to permit or inhibit the movement of the second vaporization substance from the second chamber to the channel. The user input device could also control power to the heater.

A regulator could be or include, for example, any one or more of: a wick, a valve, a pump, a spray nozzle to spray the second vaporization substance into the channel, and a mechanical feed structure such as a screw conveyor.

An apparatus could include a holder to hold the second vaporization substance in the channel. A user input device could be provided to control exposure of the holder to the channel. The user input device could also control power to the heater.

Examples of a holder include: a wick, an absorbent material, and an air permeable material, and a holder could include any one or more of these.

A vapor regulator could be provided to control a flow of the vapor from the atomizer to the heater.

In some embodiments, the first chamber and/or the second chamber includes an engagement structure to engage with a complementary engagement structure of the apparatus.

The first chamber could include an indicator of the first vaporization substance and/or the second chamber could include an indicator of the second vaporization substance. In an embodiment, the second chamber includes an indicator of the second vaporization substance, and power to the heater is controlled based on the indicator.

The heater could be or include at least one of a coil heater, a fan heater, a ceramic heater, and a quartz heater.

The first vaporization substance could be or include at least one of: a dry substance, a liquid, a gel and a wax. Similarly, the second vaporization substance could be or include at least one of: a dry substance, a liquid, a gel and a wax.

In an embodiment, the first vaporization substance includes an active substance.

The second vaporization substance could include a flavorant, for example, such as any one or more of: a terpene, an essential oil, and a volatile plant extract.

More than one secondary chambers could be provided. For example, the second chamber could be one of a plurality of chambers in fluid communication with the heater, to store respective vaporization substances for vaporization by the vapor that is heated by the heater.

Method embodiments are also contemplated. A method could involve providing a first chamber to store a first vaporization substance; providing an atomizer, in fluid communication with the first chamber, to generate vapor from the first vaporization substance by heating the first vaporization substance; providing an channel in fluid communication with the atomizer; providing a second chamber to store a second vaporization substance; providing a feeder, in fluid communication with the channel and the second chamber, to feed the second vaporization substance from the second chamber to the channel.

A method could also involve arranging the feeder downstream from the atomizer.

In an embodiment, providing the feeder involves providing an unheated atomizer.

A method could involve providing a mouthpiece in fluid communication with the channel.

Some embodiments involve arranging at least a portion of the feeder inside the channel, or inside a separate channel that is in fluid communication with the channel.

Providing the feeder could involve providing a regulator to control movement of the second vaporization substance from the second chamber to the channel. A method could also involve providing a user input device to control the regulator to permit or inhibit the movement of the second vaporization substance from the second chamber to the channel.

Providing the regulator could involve any one or more of: providing a wick, providing a valve, providing a pump, providing a spray nozzle to spray the second vaporization substance, and providing a mechanical feed structure. Providing the mechanical feed structure could involve providing a screw conveyor.

Providing a feeder could involve providing a holder to hold the second vaporization substance in the channel or the separate channel. A method could also involve providing a user input device to control exposure of the holder to the channel or the separate channel.

Providing the holder could involve any one or more of: providing a wick, providing an absorbent material, and providing an air permeable material.

A method could involve providing a vapor regulator to control a flow of the vapor from the atomizer.

Providing the first chamber and/or providing the second chamber could involve providing an engagement structure to engage with a complementary engagement structure of a vaporization device.

Providing the first chamber could involve providing an indicator of the first vaporization substance. Alternatively or in addition, providing the second chamber could involve providing an indicator of the second vaporization substance.

In an embodiment, the first vaporization substance is or includes at least one of: a dry substance, a liquid, a gel and a wax. Similarly, the second vaporization substance could be or include at least one of: a dry substance, a liquid, a gel and a wax.

The first vaporization substance could include an active substance, for example.

The second vaporization substance includes a flavorant in some embodiments, such as any one or more of: a terpene, an essential oil, and a volatile plant extract.

A method could involve providing a further chamber and a further feeder in fluid communication with the further chamber and the channel.

In some embodiments, a method involves providing a heater, in fluid communication with the atomizer, to heat the vapor from the atomizer to vaporize the second vaporization substance.

Another aspect of the present disclosure relates to a method that involves providing a first chamber to store a first vaporization substance; providing an atomizer, in fluid communication with the first chamber, to generate vapor from the first vaporization substance by heating the first vaporization substance; providing a heater, in fluid communication with the atomizer, to heat the vapor from the atomizer; and providing a second chamber, in fluid communication with the heater, to store a second vaporization substance for vaporization by the vapor that is heated by the heater.

A method could also involve providing a channel in fluid communication with the atomizer and the second chamber.

In some embodiments, a method involves providing a mouthpiece in fluid communication with the channel.

A method could involve arranging at least a portion of the heater inside the channel.

Some embodiments include providing a regulator to control movement of the second vaporization substance from the second chamber to the channel. A method could also involve providing a user input device to control the regulator to permit or inhibit the movement of the second vaporization substance from the second chamber to the channel.

Providing a regulator could involve any one or more of: providing a wick, providing a valve, providing a pump, providing a spray nozzle to spray the second vaporization substance into the channel, and providing a mechanical feed structure. Providing the mechanical feed structure could involve providing a screw conveyor.

Some embodiments involve providing a holder to hold the second vaporization substance in the channel. A method could also involve providing a user input device to control exposure of the holder to the channel.

Providing the holder could involve any one or more of: providing a wick, providing an absorbent material, and providing an air permeable material.

A method could involve providing a vapor regulator to control a flow of the vapor from the atomizer to the heater.

Either or both of providing the first chamber and providing the second chamber could involve providing an engagement structure to engage with a complementary engagement structure of a vaporization device.

Providing the first chamber could involve providing an indicator of the first vaporization substance and/or providing the second chamber could involve providing an indicator of the second vaporization substance.

The heater could include at least one of a coil heater, a fan heater, a ceramic heater, and a quartz heater.

The first vaporization substance could be or include at least one of: a dry substance, a liquid, a gel and a wax. Similarly, the second vaporization substance could be or include at least one of: a dry substance, a liquid, a gel and a wax.

The first vaporization substance could include an active substance, for example.

In some embodiments, the second vaporization substance includes a flavorant, such as any one or more of: a terpene, an essential oil, and a volatile plant extract.

A method could also involve providing a further chamber in fluid communication with the heater.

A method of use of an apparatus as disclosed herein could involve initiating vaporization of the first vaporization substance to produce a first vapor; initiating feeding of the second vaporization substance into the channel to produce a second vapor; and inhaling the first vapor and the second vapor.

Another method of use of an apparatus as disclosed herein could involve initiating vaporization of the first vaporization substance to produce a first vapor; initiating heating of the first vapor; initiating vaporization of the second vaporization substance by the first vapor that is heated by the heater, to produce a second vapor; and inhaling the first vapor and the second vapor.

Other aspects and features of embodiments of the present disclosure will become apparent to those ordinarily skilled in the art upon review of the following description.

### BRIEF DESCRIPTION OF THE DRAWINGS

For a more complete understanding of the present disclosure, reference is now made to the following description taken in conjunction with the accompanying drawings, in which:

FIG. 1 is a plan view of an example vaporization device;

FIG. 2 is an isometric view of the vaporization device in FIG. 1;

FIG. 3 is an isometric and partially exploded view of an example multi-chamber vaporization device;

FIG. 4 is a cross-sectional view of the example multi-chamber vaporization device of FIG. 3, along line A-A in FIG. 3;

FIG. 5 is a block diagram of an example vaporization device with multiple chambers in a serial configuration;

FIG. 6 is a plan view of an example vaporization device that includes a secondary chamber;

FIG. 7 is a plan and partially exploded view of the vaporization device in FIG. 6;

FIG. 8 is a plan view of the secondary chamber in FIG. 6;

FIG. 9 is another plan view of the secondary chamber in FIG. 6;

FIG. 10 is a top view of the secondary chamber in FIG. 8;

FIG. 11 is a cross-sectional view of the secondary chamber in FIG. 8, along the line B-B in FIG. 10;

FIG. 12 is a magnified portion of the cross-sectional view in FIG. 11;

FIG. 13 is another magnified portion of the cross-sectional view in FIG. 11;

FIG. 14 is a plan view of another example secondary chamber;

FIG. 15 is a top view of the secondary chamber in FIG. 14;

FIG. 16 is a cross-sectional view of the secondary chamber in FIG. 14, along the line C-C in FIG. 15;

FIG. 17 is a magnified portion of the cross-sectional view in FIG. 16;

FIG. 18 is another magnified portion of the cross-sectional view in FIG. 16;

FIG. 19 is a plan view of another example vaporization device that includes a secondary chamber;

FIG. 20 is a plan and partially exploded view of the vaporization device of FIG. 19;

FIG. 21 is a top view of the secondary chamber of FIG. 19;

FIG. 22 is a cross-sectional view of the secondary chamber of FIG. 19, along the line D-D in FIG. 21;

FIG. 22A is a plan view of another example of a secondary chamber;

FIG. 22B is another plan view of the secondary chamber in FIG. 22A;

FIG. 22C is a top view of the secondary chamber in FIG. 22A;

FIG. 22D is a cross-sectional view of the secondary chamber in FIG. 22A, along the line E-E in FIG. 22C;

FIG. 22E is a plan view of a mouthpiece that could be used with the secondary chamber in FIG. 22A.

FIG. 23 is a cross-sectional and partially exploded view of an example of engagement structures in a vaporization device;

FIG. 24 is a flow diagram illustrating a method according to an embodiment;

FIG. 25 is a flow diagram illustrating a method according to another embodiment;

FIG. 26 is a flow diagram illustrating a method according to a further embodiment; and

FIG. 27 is a flow diagram illustrating a method according to yet another embodiment.

### DETAILED DESCRIPTION

Conventional vaporization devices include a single chamber for storing a vaporization substance. However, vaporization devices with multiple chambers could be desirable. For example, multiple chambers could store different vaporization substances to create vapor mixtures with specific flavor, aromatic, and/or effect profiles. Chambers storing different vaporization substances could be fluidly connected to a channel in series, such that the vapor produced from each vaporization substance is added to the channel sequentially. The different vapors could be mixed in the air before being inhaled by a user. This is referred to herein as a serial configuration for a multi-chamber vaporization device. Chambers themselves could, but need not necessarily, be serially coupled to each other Vaporization substances could be serially fed into a channel or otherwise serially supplied for vaporization without having the chambers serially coupled together.

A serial configuration could require fewer powered components, and/or improve vapor mixing. For example, after a first vaporization substance is vaporized, additional vaporization substances could be vaporized using residual heat in the vapor that is produced from the first vaporization substance rather than using a separate heated atomizer. Therefore, fewer powered components might be required to vaporize the additional vaporization substances. Producing a vapor using heat from another vapor could also lead to better mixing of the two vapors.

For illustrative purposes, specific example embodiments will be explained in greater detail below in conjunction with the figures. It should be appreciated, however, that the present disclosure provides many applicable concepts that can be embodied in any of a wide variety of contexts. The specific embodiments discussed are merely illustrative and do not limit the scope of the present disclosure. For example, relative to embodiments shown in the drawings and/or referenced herein, other embodiments may include additional, different, and/or fewer features. The figures are also not necessarily drawn to scale.

The present disclosure relates, in part, to vaporization apparatus such as vaporization devices for vaporization substances that include substances such as cannabinoids or nicotine. However, the vaporization devices described herein could also or instead be used for other types of vaporization substances.

As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

For the purpose of this specification, the expression “cannabinoid” means a compound such as tetrahydrocannabinol (THC), cannabidiol (CBD), cannabigerolic acid (CBGA), cannabigerol (CBG), cannabigerol monomethylether (CBGM), cannabigerovarin (CBGV), cannabichromene (CBC), cannabichromevarin (CBCV), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

In some embodiments, the cannabinoid is CBD. For the purpose of this specification, the expressions “cannabidiol” or “CBD” are generally understood to refer to one or more of the following compounds, and, unless a particular other stereoisomer or stereoisomers are specified, includes the compound “Δ2-cannabidiol.” These compounds are:
(1) Δ5-cannabidiol (2-(6-isopropenyl-3-methyl-5-cyclohexen-1-yl)-5-pentyl-1,3-benzenediol);
(2) Δ4-cannabidiol (2-(6-isopropenyl-3-methyl-4-cyclohexen-1-yl)-5-pentyl-1,3-benzenediol);
(3) Δ3-cannabidiol (2-(6-isopropenyl-3-methyl-3-cyclohexen-1-yl)-5-pentyl-1,3-benzenediol);
(4) Δ3,7-cannabidiol (2-(6-isopropenyl-3-methylenecyclohex-1-yl)-5-pentyl-1,3-benzenediol);
(5) Δ2-cannabidiol (2-(6-isopropenyl-3-methyl-2-cyclohexen-1-yl)-5-pentyl-1,3-benzenediol);
(6) Δ1-cannabidiol (2-(6-isopropenyl-3-methyl-1-cyclohexen-1-yl)-5-pentyl-1,3-benzenediol);
and (7) Δ6-cannabidiol (2-(6-isopropenyl-3-methyl-6-cyclohexen-1-yl)-5-pentyl-1,3-benzenediol).

In some embodiments, the cannabinoid is THC. THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A vaporization substance may include a cannabinoid in its pure or isolated form or in a source material that includes the cannabinoid. The following are non-limiting examples of source materials that include cannabinoids: cannabis or hemp plant material (e.g., flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials that include cannabinoids are combined with water, lipids, hydrocarbons (e.g., butane), ethanol, acetone, isopropanol, or mixtures thereof.

In some embodiments, the cannabinoid is a mixture of THC and CBD. The w/w ratio of THC to CBD in the vaporization substance may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some embodiments, a vaporization substance may include products of cannabinoid metabolism, including 11-hydroxy-Δ9-tetrahydrocannabinol (11-OH-THC).

These particulars of cannabinoids are intended solely for illustrative purposes. Other embodiments are also contemplated.

As used herein, the term “terpene” (or “decarboxylated terpene”, which is known as a terpenoid) is generally understood to include any organic compound derived, biosynthetically for example, from units of isoprene. Terpenes may be classified in any of various ways, such as by their sizes. For example, suitable terpenes may include monoterpenes, sesquiterpenes, or triterpenes. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270.

In general, a vaporization substance includes one or more target compounds or components. A target compound or component need not necessarily have a psychoactive effect. One or more flavorants, such as any one or more of: terpene(s), essential oil(s), and volatile plant extract(s), may also or instead be a target compound for vaporization in order to provide flavor to a vapor flow. A vaporization substance may also or instead include other compounds or components, such as one or more carriers. A carrier oil is one example of a carrier.

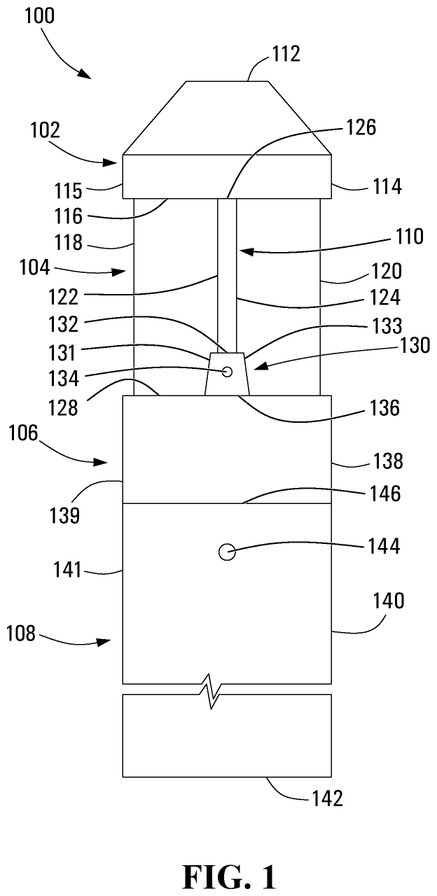

Turning now to vaporization devices in more detail, FIG. 1 is a plan view of an example vaporization device 100. In FIG. 1, the vaporization device 100 is viewed from the side. The vaporization device 100 could also be referred to as a vaporizer, a vaporizer pen, a vape pen or an electronic or “e-” cigarette, for example. The vaporizer 100 includes a cap 102, a chamber 104, a base 106 and a battery compartment 108.

The cap 102 is an example of a lid or cover, and includes a tip 112 and sidewalls 114 and 115, which are sides or parts of the same cylindrical sidewall in some embodiments. The cap 102, in addition to sealing an end of an interior space of the chamber 104, also provides a mouthpiece through which a user can draw vapor from the vaporization device 100 in some embodiments. The mouthpiece is tapered as shown in FIG. 1, and/or otherwise shaped for a user's comfort. The present disclosure is not limited to any particular shape of the cap 102.

The cap 102 could be made from one or more materials including metals, plastics, elastomers and ceramics, for example. However, other materials may also or instead be used.

In other embodiments, a mouthpiece is separate from the cap 102. For example, a cap may be connected to a mouthpiece by a hose or pipe that accommodates flow of vapor from the cap to the mouthpiece. The hose or pipe may be flexible or otherwise permit movement of the mouthpiece relative to the cap, allowing a user to orient the mouthpiece independently from the cap.

The chamber 104 is an example of a vessel to store a vaporization substance prior to vaporization. Although embodiments are described herein primarily in the context of vaporization liquids such as oil concentrates, in general a chamber may store other forms of vaporization substances, including waxes and gels for example. Vaporization substances with water-based carriers are also contemplated. A vaporization device may be capable of vaporizing water-based carriers with emulsified cannabinoids, for example. The chamber 104 may also be referred to as a container, a housing or a tank.

The chamber 104 includes outer walls 118 and 120. Although multiple outer walls are shown in FIG. 1 at 118 and 120, the chamber 104 is perhaps most often cylindrical, with a single outer wall. The outer walls 118 and 120 of the chamber 104 may be made from one or more transparent or translucent materials, such as tempered glass or plastics, in order to enable a user to visibly determine the quantity of vaporization substance in the chamber. The outer walls 118 and 120 are made from one or more opaque materials such as metal alloys, plastics or ceramics in some embodiments, to protect the vaporization substance from degradation by ultraviolet radiation, for example. The outer walls 118 and 120 of the chamber 104 may include markings to aid the user in determining the quantity of vaporization liquid in the chamber. The chamber 104 may have any of a number of different heights and/or other dimensions, to provide different interior volumes.

The chamber 104 engages the cap 102, and may be coupled to the cap, via an engagement or connection at 116. A gasket or other sealing member may be provided between the chamber 104 and the cap 102 to seal the vaporization substance in the chamber.

Some chambers are “non-recloseable” or “disposable” and cannot be opened after initial filling. Such chambers are permanently sealed once closed, and are not designed to be opened and re-sealed. Others are recloseable chambers in which the engagement at 116, between the cap 102 and the chamber 104, is releasable. For example, in some embodiments the cap 102 is a cover that releasably engages the chamber 104 and seals a vaporization substance in the chamber 104. One example of a releasable engagement disclosed elsewhere herein is a threaded engagement or other type of connection, with an abutment between the chamber 104 and the cap 102 but without necessarily an actual connection between the chamber and the cap. Such a releasable engagement permits the cap 102 to be disengaged or removed from the chamber 104 so that the chamber can be cleaned, emptied, and/or filled with a vaporization substance, for example. The cap 102 is then re-engaged with the chamber 104 to seal the vaporization substance inside the chamber.

FIG. 1 also illustrates a stem 110 inside the chamber 104. The stem 110 is a hollow tube or channel through which vapor can be drawn into and through cap 102. The stem 110 may also be referred to as a central column, a central post, a chimney, a hose or a pipe. The stem 110 includes outer walls 122 and 124, although in many embodiments the stem is cylindrical, with a single outer wall. Materials such as stainless steel, other metal alloys, plastics and ceramics may be used for stems such as the stem 110. The stem 110 couples the cap 102 via an engagement or connection 126. Similar to the engagement or connection 116, the engagement or connection 126 is a releasable engagement or connection in some embodiments, and includes a releasable engagement between the stem 110 and the cap 102. In some embodiments, the engagement 126 is in the form of, or includes, a releasable connection.

Although labeled separately in FIG. 1, the engagements at 116 and 126 are operationally related in some embodiments. For example, in some embodiments screwing the cap 102 onto the stem 110 also engages the cap with the chamber 104, or similarly screwing the cap onto the chamber also engages the cap with the stem. This is one example of a threaded connection that also releasably maintains an abutment between the chamber 104 and the cap 102 but without an actual connection between the chamber and the cap.

An atomizer 130 is provided at the base of the stem 110, inside the chamber 104. The atomizer 130 may also be referred to as a heating element, a core, or a ceramic core. The atomizer 130 includes sidewalls 131 and 133, which actually form a single cylindrical or frustoconical wall in some embodiments, and one or more wicking holes or intake holes, one of which is shown at 134. The sidewalls of the atomizer 130 may be made from a metal alloy such as stainless steel, for example. The sidewalls 131 and 133 of the atomizer 130 are made from the same material as the stem 110 in some embodiments, or from different materials in other embodiments.

The atomizer 130 engages, and may couple with, the stem 110 via an engagement 132, and with the base 106 via an engagement 136. Although the engagements 132 and 136 may be releasable, the stem 110, the atomizer 130, and the base 106 are permanently attached together in some embodiments. The atomizer sidewalls 131 and 133 may even be formed with the stem 110 as an integrated single physical component.

In general, the atomizer 130 converts the vaporization substance in the chamber 104 into a vapor, which a user draws from the vaporization device 100 through the stem 110 and the cap 102. Vaporization liquid is drawn into the atomizer 130 through the wicking hole 134 and a wick in some embodiments. The atomizer 130 may include a heating element, such as a resistance coil around a ceramic wick, to perform the conversion of vaporization liquid into vapor. A ceramic atomizer may have an integrated heating element such as a coiled wire inside the ceramic, similar to rebar in concrete, in addition to or instead of being wrapped in a coiled wire. A quartz heater is another type of heater that may be used in an atomizer.

In some embodiments, the combination of the atomizer 130 and the chamber 104 is referred to as a cartomizer.

The base 106 supplies power to the atomizer 130, and may also be referred to as an atomizer base. The base 106 includes sidewalls 138 and 139, which form a single sidewall such as a cylindrical sidewall in some embodiments. The base 106 engages, and may also be coupled to, the chamber 104 via an engagement 128. The engagement 128 is a fixed connection in some embodiments. In other embodiments the engagement 128 is a releasable engagement, and the base 106 can be considered a form of a cover that releasably engages the chamber 104 and seals a vaporization substance in the chamber 104. In such embodiments, the engagement 128 may include a threaded engagement or a threaded connection or an abutment between the chamber 104 and the base 106, for example. A gasket or other sealing member may be provided between the chamber 104 and the base 106 to seal the vaporization substance in the chamber. Such a releasable engagement enables removal or disengagement of the base 106 from the chamber 104 to permit access to the interior of the chamber, so that the chamber can be emptied, cleaned, and/or filled with a vaporization substance, for example. The base 106 is then re-engaged with the chamber 104 to seal the vaporization substance inside the chamber.

The base 106 generally includes circuitry to supply power to the atomizer 130. For example, the base 106 may include electrical contacts that connect to corresponding electrical contacts in the battery compartment 108. The base 106 may further include electrical contacts that connect to corresponding electrical contacts in the atomizer 130. The base 106 may reduce, regulate or otherwise control the power/voltage/current output from the battery compartment 108. However, this functionality may also or instead be provided by the battery compartment 108 itself. The base 106 may be made from one or more materials including metals, plastics, elastomers and ceramics, for example, to carry or otherwise support other base components such as contacts and/or circuitry. However, other materials may also or instead be used.

The combination of a cap 102, a chamber 104, a stem 110, an atomizer 130, and a base 106 is often referred to as a cartridge or “cart”.

The battery compartment 108 could also be referred to as a battery housing. The battery compartment 108 includes sidewalls 140 and 141, a bottom 142 and a button 144. The sidewalls 140 and 141, as noted above for other sidewalls, form a single wall such as a cylindrical sidewall in some embodiments. The battery compartment 108 engages, and may also couple to, the base 106 via an engagement 146. The engagement 146 is a releasable engagement in some embodiments, such as a threaded connection or a magnetic connection, to provide access to the inside of the battery compartment 108. The battery compartment 108 may include single-use batteries or rechargeable batteries such as lithium-ion batteries. A releasable engagement 146 enables replacement of single-use batteries and/or removal of rechargeable batteries for charging, for example. In some embodiments, rechargeable batteries are recharged by an internal battery charger in the battery compartment 108 without removing them from the vaporization device 100. A charging port (not shown) may be provided in the bottom 142 or a sidewall 140, 141, for example. The battery compartment 108 may be made from the same material(s) as the base 106 or from one or more different materials.

The button 144 is one example of a user input device, which may be implemented in any of various ways. Examples include a physical or mechanical button or switch such as a push button. A touch sensitive element such as a capacitive touch sensor may also or instead be used. A user input device need not necessarily require movement of a physical or mechanical element.

Although shown in FIG. 1 as a closed or flush engagement, the engagement 146 between the base 106 and the battery compartment 108 need not necessarily be entirely closed. A gap between outer walls of the base 106 and the battery compartment 108 at the engagement 146, for example, may provide an air intake path to one or more air holes or apertures in the base that are in fluid communication with the interior of the stem 110. An air intake path may also or instead be provided in other ways, such as through one or more apertures in a sidewall 138, 139, elsewhere in the base 106, and/or in the battery compartment 108. When a user draws on a mouthpiece, air is pulled into the air intake path and through a channel. In FIG. 1, the channel runs through the atomizer 130, where air mixes with vapor formed by the atomizer, and the stem 110. The channel also runs through the cap 102 in some embodiments.

The battery compartment 108 powers the vaporization device 100 and allows powered components of the vaporization device, including at least the atomizer 130, to operate. Other powered components could include, for example, one or more light-emitting diodes (LEDs), speakers and/or other elements to provide indicators of, for example, device power status (on/off), device usage status (on when a user is drawing vapor), etc. In some embodiments, speakers and/or other elements generate audible indicators such as long, short or intermittent “beep” sounds as a form of indicator of different conditions. Haptic feedback could also or instead be used to provide status or condition indicators. Varying vibrations and/or pulses, for example, may indicate different statuses or actions in a vaporization device, such as on/off, currently vaporizing, power source connected, etc. Haptic feedback may be provided using small electric motors as in devices such as mobile phones, other electrical and/or mechanical means, or even magnetic means such as one or more controlled electronic magnets.

As noted above, in some embodiments, the cap 102, the chamber 104, the stem 110, the atomizer 130, the base 106 and/or the battery compartment 108 are cylindrical in shape or otherwise shaped in a way such that sidewalls that are separately labeled in FIG. 1 are formed by a single sidewall. In these embodiments, the sidewalls 114 and 115 represent sides of the same sidewall. Similar comments apply to outer walls 118 and 120, sidewalls 131 and 133, outer walls 122 and 124, sidewalls 138 and 139, sidewalls 140 and 141, and other walls that are shown in other drawings and/or described herein. However, in general, caps, chambers, stems, atomizers, bases and/or battery compartments that are not cylindrical in shape are also contemplated. For example, these components may be rectangular, triangular, or otherwise shaped.

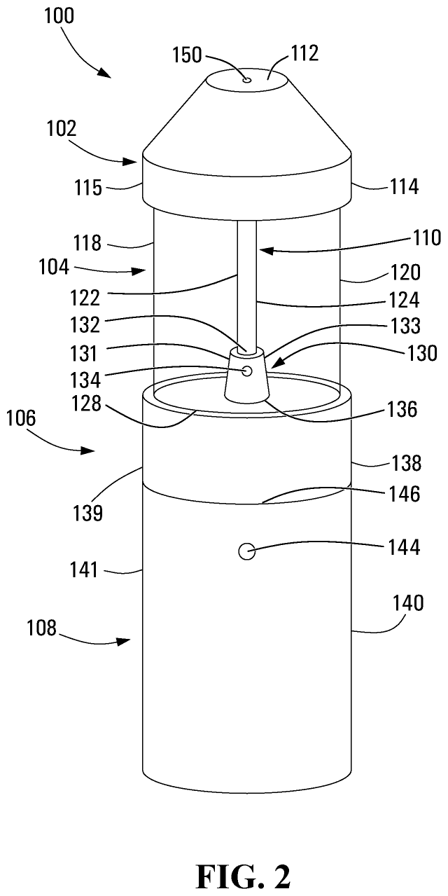

FIG. 2 is an isometric view of the vaporization device 100. In FIG. 2, the cap 102, the chamber 104, the stem 110, the atomizer 130, the base 106 and the battery compartment 108 are illustrated as being cylindrical in shape. As noted above, this is not necessarily the case in other vaporization devices. FIG. 2 also illustrates a hole 150 through the tip 112 in the cap 102. The hole 150 is coupled to the stem 110 through a channel in the cap 102. The hole 150 allows a user to draw vapor through the cap 102. In some embodiments, a user operates the button 144 to vaporize a vaporization substance for inhalation through the cap 102. Other vaporization devices are automatically activated, to supply power from the battery compartment 108 to powered components of the vaporization device when a user inhales through the hole 150. In such embodiments, a button 144 need not be operated to use a vaporization device, and need not necessarily even be provided at all.

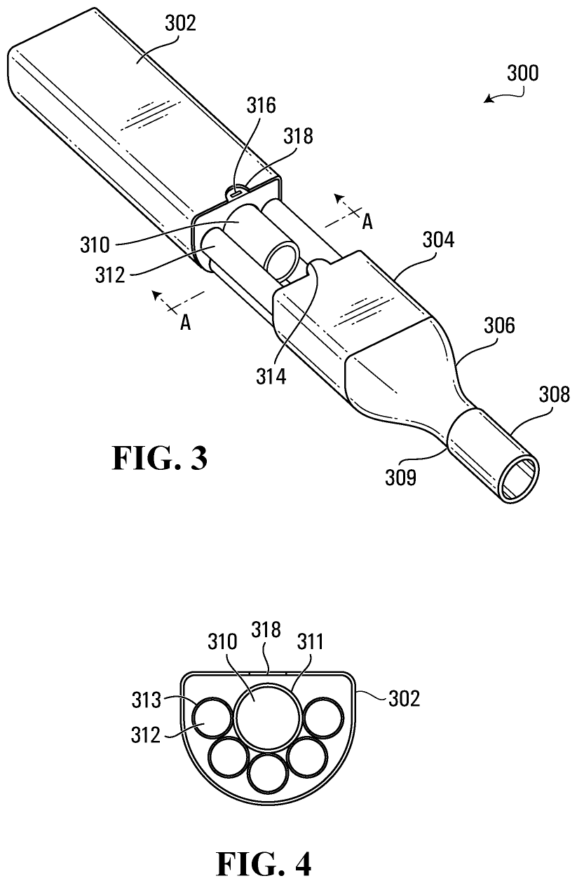

FIG. 3 is an isometric and partially exploded view of an example multi-chamber vaporization device, and FIG. 4 is a cross-sectional view of the example multi-chamber vaporization device along line A-A in FIG. 3. The vaporization device 300 has a multi-part body, with a main body 302 and a removable cover 304. The main body 302 and the cover 304 could be made from the same material(s) or different materials, including one or more of metals, plastics, elastomers and ceramics, for example. However, other materials could also or instead be used.

The main body 302 and the cover 304 include compartments to receive vaporization substance chambers 312 and a channel 310. The compartments in the main body 302 are shown at 311, 313 in FIG. 4, and the cover 304 also includes such compartments. The cover 304 tapers at 306 to a mouthpiece 308 in the example shown, and the mouthpiece is in fluid communication with the channel 310. The main body 302 could at least partially carry or otherwise support components such as the channel 310 and the chambers 312 as shown, and other components such as one or more batteries, electrical contacts, and/or circuitry. Similarly, the cover 304 could at least partially carry or otherwise support components such as the channel 310 and the chambers 312, as well as the mouthpiece 308.

Various channels such as the channel 310 enable fluid flow through a vaporization apparatus such as a vaporization device, or at least parts thereof. Such fluid may include air, at an intake side of an atomizer for example, or mixture of air and vapor upstream of an atomizer when the atomizer is operating to vaporize a vaporization substance. Fluid flow channels may also be referred to as air channels, but are referenced herein primarily as channels.

The mouthpiece 308 could be made from the same material(s) as the remainder of the cover 304, and could even be integrated with the cover. In the embodiment shown, the mouthpiece 308 engages with the remainder of the cover 304 at an engagement or connection 309. This engagement or connection 309 could be fixed, which might be preferable in embodiments in which the mouthpiece 308 is cylindrical as shown. In other embodiments, a rotatable or otherwise movable engagement or connection 309 might be preferred, so that a user can position the mouthpiece 308 in any preferred orientation relative to the main body 302 and/or the remainder of the cover 304.

Materials such as stainless steel, other metal alloys, plastics and ceramics could be used for the channel 310.

The chambers 312 could be made, at least in part, from one or more materials such as tempered glass, plastics, metal alloys, and/or ceramics. The chambers 312 could be substantially similar to chamber 104 shown by way of example in FIGS. 1 and 2, and could be coupled to other parts that are made from different materials. In some embodiments, the chambers 312 are cartridges that each include a base, an atomizer, a stem, and a cap.

The cover 304 is removable or releasable from the main body 302. In the example shown in FIG. 3, a tab 314 on the cover 304 could be provided with a protrusion on its inner surface, to engage with a groove or slot 316 in the main body 302 when the vaporization device 300 is assembled or closed. This is an example of a releasable engagement between the main body 302 and the cover 304. The cover 304 could be removed, to install or remove chambers 312 and/or for cleaning the device 300 for example, by pulling the cover 304 away from the main body 302 with sufficient force to release the protrusion on the tab 314 from the slot or groove 316. Removal of the cover 304 in the embodiment shown could also or instead involve prying the tab 314 away from the slot or groove 316 to release the tab protrusion and allow the cover to be removed.

The main body 302 could include a structure 318 to accommodate the tab 314, so that the outer surface of the tab is flush with the outer surface of the main body when the device 300 is assembled. The structure 318 could be larger than the tab 314 in some embodiments, to provide clearance for a user to insert a fingernail or tool to pry the tab away from the slot or groove 316 when the cover 304 is to be removed.

In operation, one or more batteries inside the main body 302 provide power to one or more atomizers and/or one or more heaters, which vaporize one or more vaporization substances from multiple chambers 312. Any of various arrangements or implementations are possible, and examples are disclosed herein.

It should be appreciated, however, that the example device 300 is solely for the purpose of illustration. Other embodiments are also contemplated. For example, the channel 310 need not be a separate component and could be integrated or integral with the main body 302 and/or the cover 304. Each chamber 312 could include a stem as shown in FIGS. 1 and 2, and the cover 304 could then include a manifold to fluidly couple each stem to the mouthpiece 308 without the channel 310. The channel 310 and/or the chambers 312 could be accommodated entirely within the main body 302, in which case the cover 304 need not include compartments to receive part of each chamber. Compartments could be implemented in any of various ways, and not only as the bores shown at 311, 313 in FIG. 4. Multiple engagement structures such as the tab 314 and the slot or groove 316 could be provided. Other types of connection or engagement between a main body and a cover, such as a magnetic connection, are also possible. Different shapes or layouts could be implemented, to have a central channel with compartments or structures to accommodate chambers around the central channel, for example. A multi-chamber vaporization device with a hexagonal cross-sectional shape, for example, could accommodate six cartridges or chambers around a central channel or mixing channel. At least certain shapes could be suitable for other types of releasable engagement between a main body and a cover, such as a threaded engagement for a cylindrical vaporization device.

With multiple vaporization substances available in a multi-chamber vaporization device, more than one vaporization substance could be vaporized for inhalation. For example, as disclosed herein, multiple chambers could be fluidly connected to a channel in a series configuration. In FIGS. 3 and 4, for example, vaporization substances from different chambers 312 could be fed into the channel 310 for vaporization at different points or positions along the channel. The channel itself could be coupled to an in-line “pull through” chamber through which a vapor that is produced from a vaporization substance in another chamber is drawn.

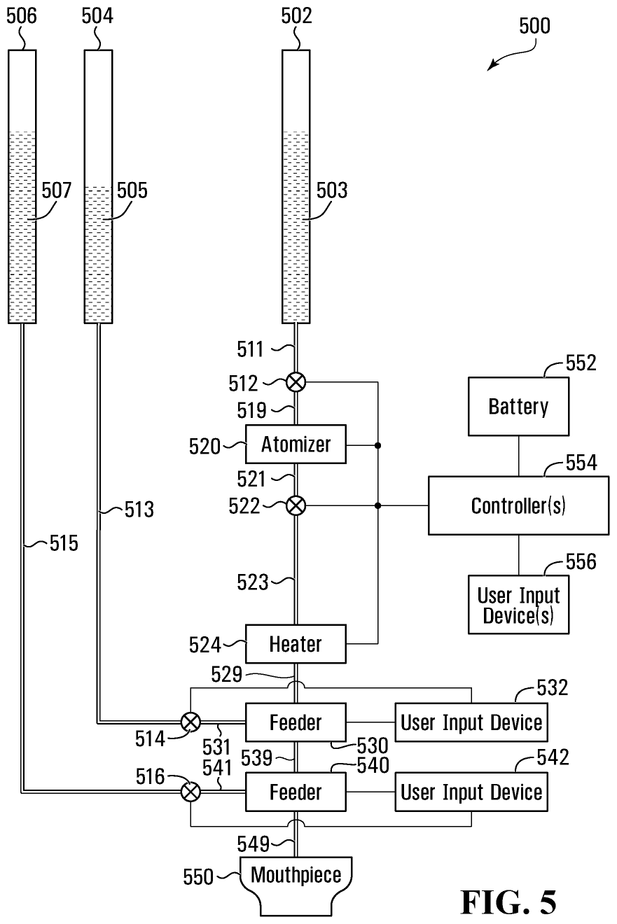

FIG. 5 is a block diagram of an example vaporization device 500 including multiple chambers 502, 504, 506 in a serial configuration, at least in terms of feeding vaporization substances to a channel. The multiple chambers 502, 504, 506 of FIG. 5 are provided to store respective vaporization substances 503, 505, 507. The chamber 502 is in fluid communication with an atomizer 520 to generate vapor from the vaporization substance 503 by heating the vaporization substance. The vapor produced by the atomizer 520 is fed into an channel 521. The chambers 504, 506 are in fluid communication with respective feeders 530, 540. The feeders 530, 540 are provided to feed respective vaporization substances 505, 507 from the chambers 504, 506 to channels 539, 549. In FIG. 5, the chamber 502 could be considered to be a primary chamber, as this chamber is fluidly connected to the channel 521 upstream of the other chambers with respect to a direction of fluid flow, which is downward in the example shown in FIG. 5. The chambers 504, 506, on the other hand, could be considered to be secondary chambers, as these chambers are fluidly connected to the channels 539, 549, which are downstream of channel 521 with respect to the direction of fluid flow.

The feeders 530, 540 do not contain heaters to vaporize the vaporization substances 505, 507. Instead, the feeders 530, 540 could include an unpowered or unheated atomizer to vaporize one or more of the vaporization substances 505, 507. The feeders 530, 540 could also or instead vaporize the vaporization substances 505, 507 using heat from the vapor produced by the atomizer 520. A heater 524, which is in fluid communication with the atomizer 520, could heat the vapor from the atomizer to help vaporize the downstream or secondary vaporization substances 505, 507. The vapors produced from any or all of the vaporization substances 503, 505, 507 could be mixed for inhalation by a user through a mouthpiece 550.

The chambers 502, 504, 506 could be similar to the chamber 104 described above with reference to FIGS. 1 and 2. Any or all of the chambers 502, 504, 506 could include engagement structures to engage with complementary engagement structures of the example device 500. These engagement structures could limit the example device 500 to certain types of chambers, and examples of such engagement structures are disclosed elsewhere herein.

The chamber 502 is in fluid communication with a valve 512 through a channel 511. A feeder 530 and/or 540 could also or instead include a valve, and for illustrative purposes valves 514 and 516 are separately shown in FIG. 5 Each of the chambers 504 and 506 is in fluid communication with a respective valve 514 and 516 through a respective channel 513 and 515.

The valves 512, 514, 516 in the device 500 are examples of regulators to control movement of the vaporization substances from their respective chambers. Other forms of regulators include, for example, wicks, pumps, and mechanical feed structures such as screw conveyors. Regulators could also include spray nozzles to spray one or more vaporization substances into a channel, which creates a mist or vapor. For example, the valve 514 could also or instead include a spray nozzle to spray the vaporization substance 505 into the channel 539. This spray nozzle might generate a vapor that is suitable for inhalation, in which case the spray nozzle could be considered an unheated atomizer. Alternatively, the spray nozzle could generate a mist that is converted into a vapor using heat from the vapor produced by the atomizer 520 and/or the heater 524. Specific examples of spray nozzles are provided elsewhere herein.

Regardless of the type(s) of regulators in a multi-chamber device, the regulators may be useful in providing a measure of dosage control. Different vaporization substances could have different levels of active ingredients, for example, and overall dosage of active ingredients in a mixture of vaporization substances could be controlled by controlling the regulators.

The valve 512 is in fluid communication with the atomizer 520 through channel 519. The atomizer 520 could be similar to the atomizer 130 described above with reference to FIGS. 1 and 2. In some embodiments, the valve 512 could be integrated with the atomizer 520 in a single component. The valve 512 controls the movement of the vaporization substance 503 to the atomizer 520, which generates a vapor by heating the vaporization substance.

The atomizer 520 is in fluid communication with a vapor valve 522 through channel 521. The vapor valve 522 is an example of a vapor regulator, which is provided to control a flow of the vapor from the atomizer. The vapor valve 522 is in fluid communication with the heater 524 through channel 523. The heater could include, for example, a coil heater, a fan heater, a ceramic heater, and/or a quartz heater. The heater 524 is provided to heat the vapor from the atomizer 520. This heated vapor could then be used to vaporize the vaporization substance 505 and/or the vaporization substance 507. At least a portion of the heater 524 could be inside of the channel 529 to directly heat the vapor. The heater 524 could also or instead be outside of the channel 529 to conduct heat through the walls of the channel to heat the vapor.

The valve 512, the atomizer 520, the vapor valve 522 and/or the heater 524 are controlled by one or more controllers 554. A controller at 554 could be implemented, for example, using hardware, firmware, one or more components that execute software stored in one or more non-transitory memory devices (not shown), such as a solid-state memory device or a memory device that uses movable and/or even removable storage media. Microprocessors, Application Specific Integrated Circuits (ASICs), Field Programmable Gate Arrays (FPGAs), and Programmable Logic Devices (PLDs) are examples of processing devices that could be used to execute software.

A battery 552 and one or more user input devices 556 are coupled to the controller(s) 554. The user input device(s) 556 could include switches, sliders, dials, and/or other types of input device that enable a user to control any of various aspects or parameters of the valve 512, the atomizer 520, the vapor valve 522 and/or the heater 524. Other input device examples are disclosed elsewhere herein, with reference to the button 144 in FIGS. 1 and 2, for instance.

The battery 552 provides power to the controller(s) 554, which could then provide power to other components of the example device 500. The valve 512 could be controlled in this type of implementation by controlling power to the valve. For example, the valve 512 could be normally closed when not supplied with power, and opened when powered. In other embodiments, power and control are implemented separately. Other control mechanisms are also possible. However, not all types of regulators are necessarily controlled. A wick, for example, draws a vaporization substance from a chamber to an atomizer for vaporization, but the wick itself is not controlled.

A controller at 554 also controls and supplies power to the atomizer 520, and could provide on-off power control based on operation of a power button or switch at 556 or a user inhaling on the device 500, for example. In some embodiments, different voltages and/or currents could be supplied to the atomizer 520 to enable the atomizer to provide different temperatures for vaporization. This type of power control, which could be considered a form of temperature control, could be provided through a user input device 556, and/or based on sensing the type of chamber 502 currently installed in the device 500. For example, the chamber 502 could include an indicator of its vaporization substance 503. Using this indicator, a controller 554 could determine what vaporization temperature is appropriate for the vaporization substance 503, and control the power delivered to the atomizer 520 accordingly. The voltage, current, and/or power supplied to the atomizer 520 could also or instead be controlled based on a desired flow or quantity of vapor produced by the atomizer, which could be selected or otherwise controlled using a user input device 556, for example.

A controller at 554 further controls and powers the heater 524. This control could be similar to the control of the atomizer 520 discussed above. In some embodiments, different voltages and/or currents could be supplied to the heater 524 to heat the vapor produced by the atomizer 520 to any of various temperatures. These temperatures could be set by a user input device 556, or determined based on the vaporization temperatures of the vaporization substance 505 and/or the vaporization substance 507. Any or all of the chambers 504, 506 could include indicators of their respective vaporization substances 505, 507, and the power to the heater 524 could be controlled based on one or more of these indicators. For example, a controller 554 could control the heater 524 to heat the vapor produced by the atomizer 520 to an appropriate temperature for vaporizing the vaporization substance 505 and/or the vaporization substance 507. When no vaporization of the vaporization substance 505 or the vaporization substance 507 is desired, the power supplied to the heater 524 could be turned off. The power supplied to the heater 524 could also be turned off if the vapor temperature produced by the atomizer 520 is sufficient to vaporize the vaporization substance 505 and/or the vaporization substance 507.

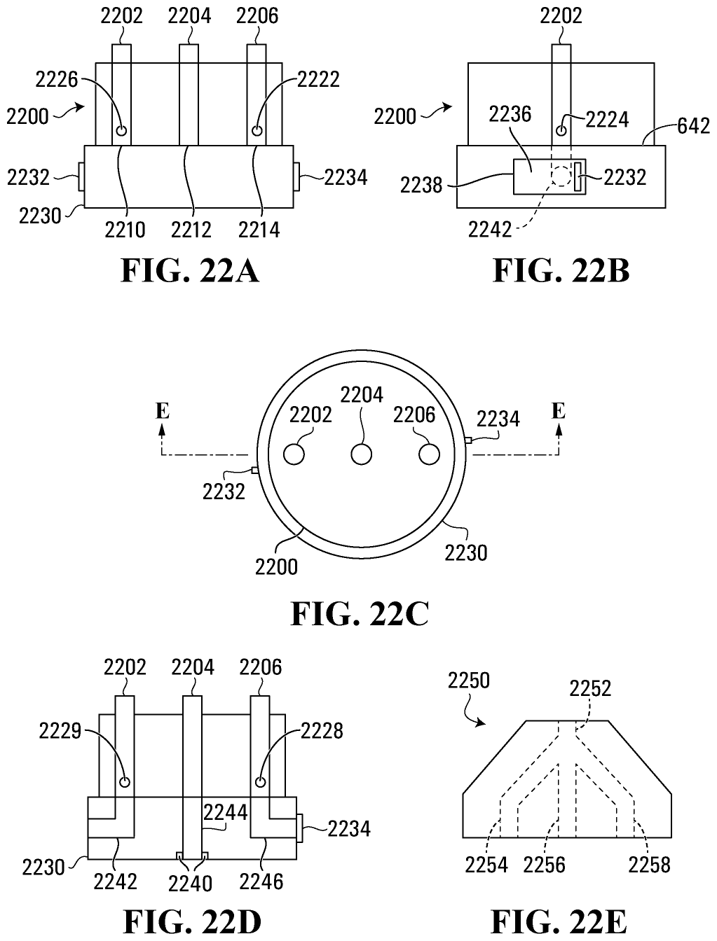

The heater 524 is in fluid communication with the feeder 530 through a channel 529. The feeder 530 is also in fluid communication with the valve 514 through channel 531, and in fluid communication with the feeder 540 through channel 539. In some embodiments, the valve 514 could be integrated with the feeder 530 in a single component. The feeder 530 could be coupled to the channels 529 and 539 in any number of ways. For example, at least a portion of the feeder 530 could be inside the channel 529 and/or the channel 539. The feeder 530 could instead be coupled to the channels 529, 539 through a separate channel instead, and an embodiment with separate feeder channels is discussed below with reference to FIGS. 22A to 22E.

The feeder 530 is provided to feed the vaporization substance 505 to the channel 539, where it could be vaporized. Feeding could include allowing the vaporization substance to flow into the channel 539, directly or through a separate channel, and/or pumping the vaporization substance into the channel, again directly or through a separate channel, for example. Examples of feeders are disclosed elsewhere herein. The feeder 530 could vaporize the vaporization substance 505 without there being an integrated heater or other source of heat within the feeder. In this sense, the feeder 530 could be considered to be or include an unheated atomizer. In some embodiments, the vaporization substance 505 that is fed into the channel 539 by the feeder 530, directly or through a separate channel, is vaporized by heat from the vapor produced by the atomizer 520. The heater 524 could increase the temperature of the vapor produced by the atomizer 520, to aid in the vaporization of the vaporization substance 505. For example, the heater 524 could increase the temperature of the vapor produced by the atomizer 520 to reach the vaporization temperature of the vaporization substance 505. The vapor produced from the vaporization substance 505 could combine with the vapor from the vaporization substance 503 to form a vapor mixture.

One or more user input devices 532 could be coupled to the feeder 530 and the valve 514 as shown. A user input device 532 could control the valve 514 to permit or inhibit the movement of the vaporization substance 505 from the chamber 504 to the channel 539. A user input device 532 could further control other features or aspects of the feeder 530. For example, the feeder 530 could include a holder to hold the vaporization substance 505 in the channel 539, or in a separate channel. The holder could include a wick, an absorbent material such as cotton, and/or an air permeable material such as a membrane or filter, for example. A user input device 532 could control exposure of the holder, or at least the vaporization substance that the holder holds, to the channel 539 or a separate channel, thereby controlling the vaporization of the vaporization substance 505. In some embodiments, the same user input device 532 could be further coupled to the heater 524 to control power to the heater. That user input device 532 could turn on the heater 524 at the same time the valve 514 is opened to allow the vaporization substance 505 to flow into the holder (feeder 530) for vaporization, for example.

In the example device 500, no battery or other source of external power is coupled to the user input device 532. Therefore, the control of the valve 514 and/or the feeder 530 could be manual. For example, the user input device 532 could include a switch that is mechanically coupled to the valve 514 to control the valve. The same switch, and/or another user input device, could also be mechanically connected to the feeder 530 to engage or disengage a holder and the channel 539 or a separate channel, for example. In other embodiments, a power source and/or controller could be coupled to the user input device 532 to control the valve 514 and the feeder 530.

The feeder 540 is in fluid communication with the feeder 530 through the channel 539 in the example shown. The feeder 540 is also in fluid communication with the valve 516 through channel 541. The feeder 540 could operate in much the same manner as the feeder 530, to feed the vaporization substance 507 from the chamber 506, directly or through a separate channel, to a channel 549, where it could be vaporized. The feeder 540 and valve 516 are coupled to a user input device 542, which could be similar to the user input device 532. However, user input devices need not necessarily be specific to one feeder. For example, a single user input device could be used to control both valves 514, 516 and both feeders 530, 540.

The mouthpiece 550 is in fluid communication with the channel 549. A user may draw vapor from the device 500 through the mouthpiece 550. As noted above, this vapor could include vapor produced from any or all of the vaporization substances 503, 505, 507. Mixing of vapors produced from different vaporization substances could occur in the mouthpiece 550. The mouthpiece 550 could be provided in the form of a cap, such as cap 102 in FIGS. 1 and 2.

A specific example of a vaporization device 500 is shown in FIG. 5. Other embodiments are also contemplated. For example, any or all of the valves 512, 514, 516, and the vapor valve 522 could be excluded in other vaporization devices. The heater 524 could also or instead be excluded. Alternatively, additional heaters could be provided, for example between the feeder 530 and the feeder 540. More or fewer chambers in fluid communication with respective feeders are also contemplated. In some embodiments, the number of chambers/feeders could be configurable, such that a user can add or remove chambers/feeders as desired. FIG. 5 illustrates an example in which the feeders 530, 540 are downstream from the atomizer 520. However, one or more feeders could also or instead be upstream of an atomizer. For example, a spray nozzle could feed a vaporization substance into a channel that flows into an atomizer, where another vaporization substance is heated and vaporized.

Although the channels 521, 523, 529, 539 and 549 are all illustrated separately, these channels could instead form a single continuous channel from the atomizer 520 to the mouthpiece 550. At least a portion of the vapor valve 522, heater 524, and/or feeders 530, 540 could be inside of this continuous channel.

A vaporization device could include a cooler in some embodiments, to reduce the temperature of the final vapor before inhalation, so that the vapor is pleasurable to inhale. The cooler could be active, using thermoelectric cooling for example, or passive, using a heat sink for example. The cooler could be provided in fluid communication with the channel 549 upstream of the mouthpiece 550, and/or within the mouthpiece.

In an embodiment, a heat sink or even multiple heat sinks could be removably installed in the channel 549, in the mouthpiece 550, and/or between the channel and the mouthpiece. The heat sink(s) could be held in place magnetically or otherwise. In some embodiments, a heat sink is removable so that it can be cooled by refrigeration before use.

Alternatively or in addition, the channel 549 and/or the mouthpiece 550 could provide a cooling effect. For example, the channel 549 could be lengthened and be in the form of a hose, for example, to provide time for vapor to cool before it reaches the mouthpiece 550. The channel could also or instead be made from or at least include materials with a high thermal conductivity, such as copper, to help cool the vapor.

Cooling could also or instead be provided by intake air. Additional intake air could be allowed into the mouthpiece 550, into the channel 549, and/or into part of a vaporization device channel upstream from the channel 549. Control of intake air flow could be manual and/or automatic. A user could manually control intake air flow by operating one or more valves and/or other air flow control component(s) to provide a desired temperature at the mouthpiece 550. Automatic control could be responsive to one or more temperature sensors to sense temperature of air in a channel and provide measurements and/or other signals to control operation of one or more air flow control components. Another intake air control option would be to control one or more air flow control components based on operation of a vapor heater such as 524. For example, a vapor heater such as 524 and one or more intake air flow control components could be operated or controlled together, to increase intake air flow when the vapor heater is in operation and to decrease intake air flow when the vapor heater is not in operation.

The vaporization substances 503, 505, 507 could be in the form of dry substances, liquids, gels and/or a waxes. The vaporization substances 503, 505, 507 could have any of various effects. For example, some vaporization substances could include one or more active ingredients that have a psychoactive effect, whereas others could include flavorants such as any one or more of: terpenes, an essential oil, and a volatile plant extract. In some embodiments, the vaporization substance 503 contains an active substance, and the vaporization substances 505 and 507 include flavorants. A user could, using the user input devices 532, 542, 556, selectively vaporize the active substance using the atomizer 520 and the terpenes using the feeders 530, 540, creating a controllable mixture of vapors produced from the vaporization substances 503, 505, 507. This mixture could be tuned for a specific effect, flavor and/or aromatic profile desired the by the user.

The desired profile could be set in one or more of the user input devices 532, 542, 556. For example, the user input devices 532, 542, 556 could control the valves 512, 514, 516, the atomizer 520, the vapor valve 522, the feeders 530, 540 and/or the heater 524 to achieve a desired flavor profile. The flavorants in the vaporization substances 505, 507 could have a low vaporization temperature relative to the active substance in the vaporization substance 503. As such, the residual heat in the vapor produced by the atomizer 520 could be sufficient to vaporize the flavorants. The heater could also or instead be used to increase the temperature of the vapor in the channel 523 to promote vaporization of the vaporization substances 505, 507. A passive atomizer such as a spray nozzle in the valves 514, 516 and/or the feeders 530, 540 could also or instead cause or assist in vaporization of the vaporization substances 505, 507.

FIG. 5 illustrates a general example of a vaporization device with multiple chambers in a serial configuration. Specific examples of vaporization devices with multiple chambers in serial configurations will now be discussed.

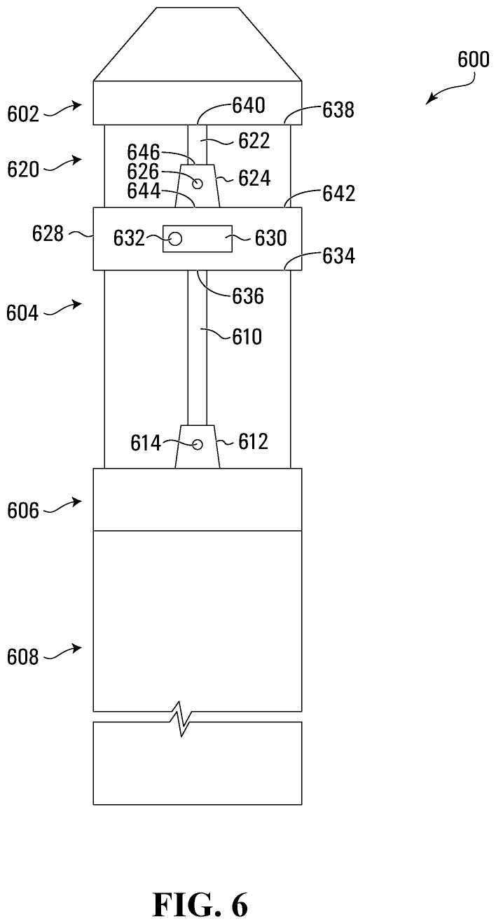

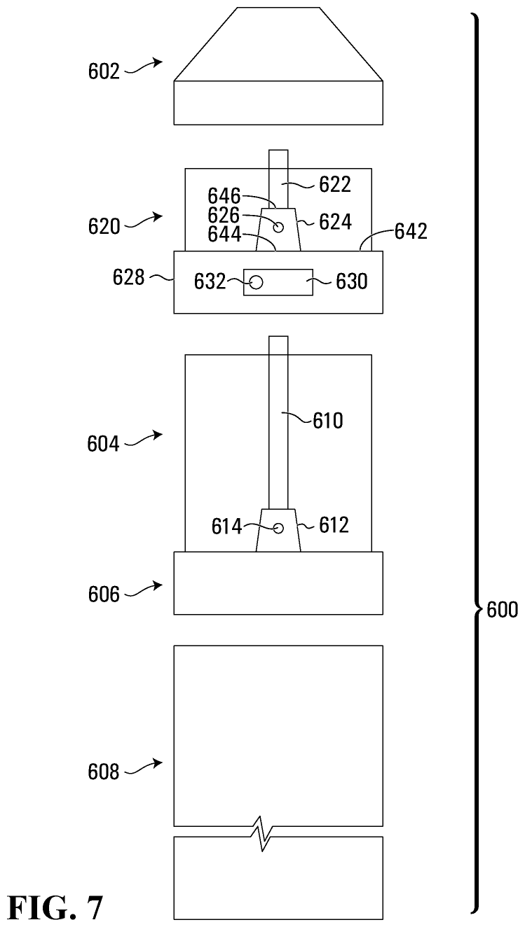

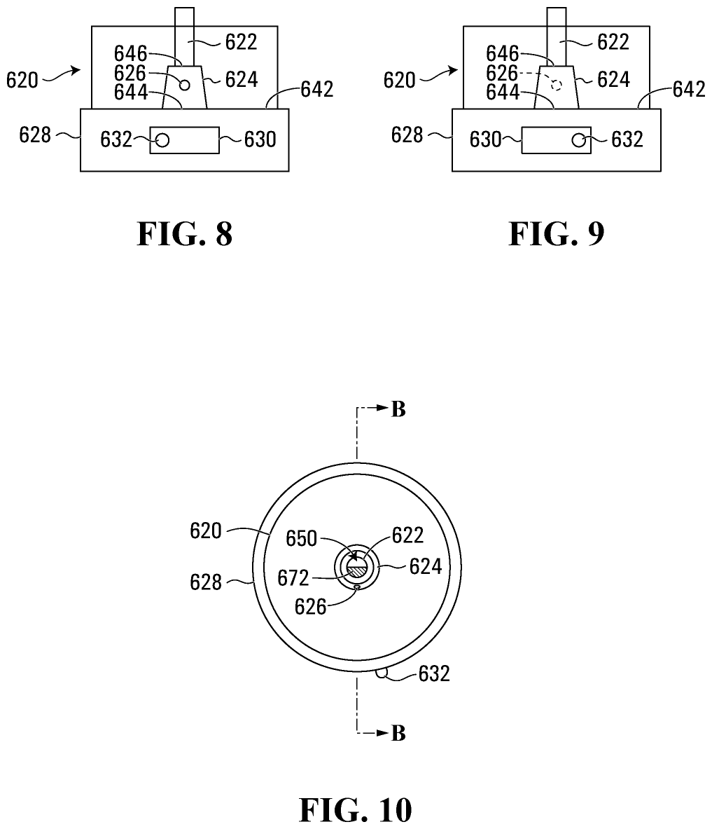

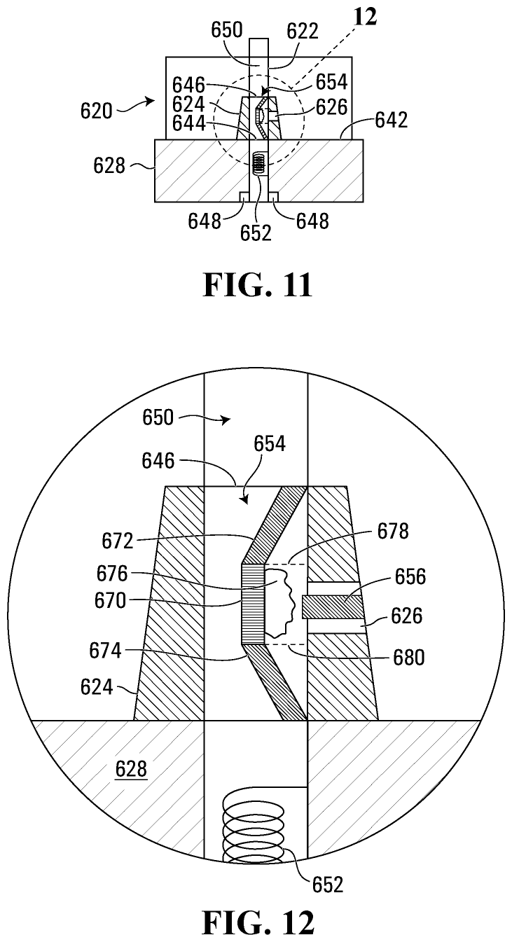

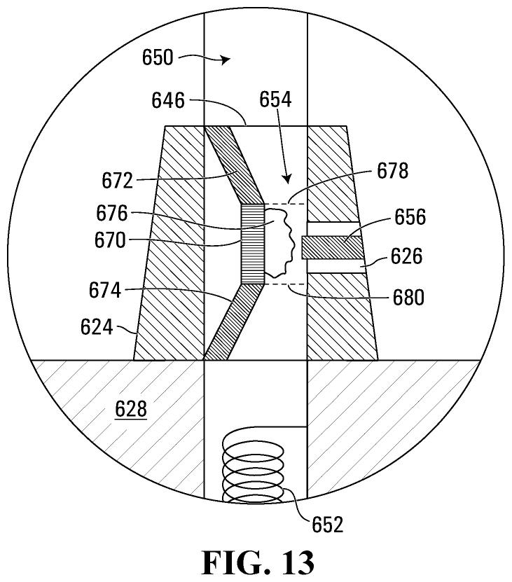

FIG. 6 is a plan view of an example vaporization device 600 that includes a secondary chamber 620, FIG. 7 is a plan and partially exploded view of the vaporization device 600, FIGS. 8 and 9 are plan views of the secondary chamber 620, FIG. 10 is a top view of the secondary chamber 620, and FIG. 11 is a cross-sectional view of the secondary chamber 620, along the line B-B in FIG. 10. FIGS. 12 and 13 are magnified portions of the cross-sectional view in FIG. 11, and provide a more detailed view of region 12 in FIG. 11. Various features referenced in the description below are shown in one or more of these drawings.

The vaporization device 600 includes, in part, a cap 602, a primary chamber 604, a base 606, a battery compartment 608, a stem 610, an atomizer 612, and an intake hole 614.

These components could be similar to the cap 102, chamber 104, base 106, battery compartment 108, stem 110, atomizer 130 and intake hole 134 discussed above with reference to FIGS. 1 and 2.

At its top end in the view shown in FIG. 6, the primary chamber 604 is engaged with a base 628 via an engagement 634, and the stem 610 is engaged with the base 628 via an engagement 636. As shown in FIG. 11, the base 628 includes notches 648, which could be an annular groove, to accommodate and/or engage with the distal end of the stem 610, relative the atomizer 612. The base 628 includes a user input device 630, which includes a switch 632. The base 628 also engages the secondary chamber 620, via an engagement 642, and to a feeder 624 via an engagement 644. The feeder 624 includes an intake hole 626. The feeder 624 is engaged with a stem 622 via an engagement 646. The secondary chamber 620 engages with the cap 602 via an engagement 638, and the stem 622 also engages with the cap 602 via an engagement 640.

At least the secondary chamber 620 and the stem 622 could be similar to example chambers and stems disclosed elsewhere herein. The base 628 includes the user input device 630, which might not be found in other embodiments, and engages with the primary chamber 604 and the stem 610 instead of to a battery compartment, but could otherwise be similar to bases in other embodiments. The feeder 624 could also be substantially similar in construction to atomizers in other embodiments, but without a heater or without the atomizer heater being powered.

Regarding the base 628, at its upper end in FIG. 6 the base 628 could engage with the secondary chamber 620 and the feeder 624 in a similar manner as the base 606 engages with the primary chamber 604 and the atomizer 612. At its lower end in FIG. 6, the base 628 could engage with the primary chamber 604 and the stem 610 in a similar manner as the cap 602 engages with the secondary chamber 620 and the stem 622. Examples of chamber/stem/cap and chamber/atomizer/base engagements are also disclosed elsewhere herein. Other engagements are possible as well. An adapter or “intermediate” base could have a chamber/stem type engagement on one side to engage a chamber and stem and a base/battery compartment type engagement on an opposite side, so that two cartridges with standard chamber and stem designs and standard base connectors could be arranged in a serial configuration as shown in FIG. 6 using such an adapter.

The primary chamber 604 and/or the secondary chamber 620 could be recloseable or non-recloseable. As such, any one or more of the engagements 634, 636, 638, 640, 642, 644, 646 could be releasable or non-releasable. Examples of releasable engagements and non-releasable engagements are provided elsewhere herein.

Chambers 604, 620 could be of the same size, or could have different sizes. The primary chamber 604 stores a vaporization substance, and is in fluid communication with the atomizer 612 through the intake hole 614. The atomizer 612 generates a vapor from this vaporization substance by, for example, heating the vaporization substance. The stem 610 provides a channel in fluid communication with the atomizer 612. Vapor produced in the atomizer 614 flows through this channel. The channel is in fluid communication with a channel 650, which is provided in the base 628, the feeder 624 and the stem 622.

The chamber 620 stores another vaporization substance. The feeder 624, which is downstream from the atomizer 612 relative to a direction of air flow during use of the vaporization device 600, is in fluid communication with the secondary chamber 620 and the channel 650. The feeder 624 could be made from plastics, ceramics, and/or metals alloys, for example. A portion of the feeder 624 is provided inside the channel 650. The feeder 624 is provided to feed the vaporization substance stored in the chamber 620 to the channel 650. After the vaporization substance is fed into the channel 650, it could be vaporized using heat from the vapor produced by the atomizer 612. In this sense, the feeder 624 could be considered an unpowered or unheated atomizer.

The cap 602, which could include a mouthpiece, is also in fluid communication with the channel 650.

The feeder 624 includes a wick 656 and a holder 654 in an embodiment. The wick 656 is an example of a regulator to control movement of the vaporization substance from the chamber 620 to the channel 650. The feeder 624 could also include a valve (not shown), operatively coupled to the user input device 630, to control movement of the vaporization substance from the chamber 620 to the channel 650. When movement of the vaporization substance from the chamber 620 to the channel 650 is desired, for example when a user wishes to vaporize the vaporization substance, the valve could be controlled by the user input device 630 to open the intake hole 626 of the feeder 624. However, when movement of the vaporization substance from the chamber 620 to the channel 650 is not desired, for example when a user does not wish to vaporize the vaporization substance, the valve could be controlled by the user input device 630 to close the intake hole 626 of the feeder 624.

This operation of the valve is perhaps best illustrated in FIGS. 8 and 9. In FIG. 8, the switch 632 is towards the left of the user input device 630, and the intake hole 626 is open. In FIG. 9, the switch 632 is towards the right of the user input device 630, and the intake hole 626 is closed, as illustrated by dashed lines around the intake hole. In this example, the user input device 630 controls the valve to permit or inhibit the movement of the vaporization substance from the secondary chamber 620 to the channel 650. Although FIGS. 8 and 9 illustrate a binary valve with only opened and closed operating states or positions, the valve could also or instead be variable to continuously control or adjust the movement of the vaporization substance from the secondary chamber 620 to the channel 650. At least some other types of regulator could be controlled by a user input device, to provide on/off vaporization substance flow control, and possibly more granular and even continuous flow control.

Valves and wicks could be suitable regulators for liquid vaporization substances, for example. However, in the case of solid vaporization substance for example, mechanical feed structures such as a screw conveyor could be used as a form of regulator.

The holder 654 is provided to hold the vaporization substance in the channel 650. This holder could be useful in providing continuous and consistent supply of vaporization substance to the channel 650, such that the vaporization experience is consistent for a user.

The holder 654 is perhaps best viewed in FIGS. 12 and 13. The holder includes a support 670, two flaps 672, 674, an absorbent material 676, and two air permeable membranes 678, 680. In the example shown, a vaporization substance moves from the chamber 620 to the channel 650 through the wick 656. As shown in FIGS. 12 and 13, a portion of the wick 656 is inside the channel 650, and therefore the wick could hold the vaporization substance in the channel. As such, the wick 656 could be considered to be part of a holder, and could be implemented without additional components such as the other components shown inside the channel 650 in FIG. 12, for example.

The absorbent material 676 is provided in some embodiments, and is coupled to and supported by the support 670 in the example shown. The support 670 could be coupled to the inner walls of the channel 650, to support the absorbent material 676 and the flaps 672, 674. The absorbent material 676 could absorb some or all of the vaporization substance that enters the channel 650. Although FIG. 12 and other drawings illustrate a gap between the absorbent material and the wick 656, the absorbent material could be wrapped around or otherwise in contact with part of the wick.

A vaporization substance that is absorbed by the absorbent material 676 could be held by the absorbent material until it is vaporized. The absorbent material 676 could include any material that will absorb vaporization substances, such as cotton, paper or sponge.

The air permeable membranes 678, 680, which are formed from any air permeable material that is impermeable to the vaporization substance in chamber 620, could prevent unintentional dripping or leaks from the wick 656 or the absorbent material 676 from escaping the holder 654. Dripping could, for example, foul parts of the vaporization device 600, such as the cap 602 and/or the channel 650. Dripping could also create an undesirable “wet” sensation when a user inhales of the vaporization device 600. Thus, the air permeable membranes 678, 680 could better confine the vaporization substances to the holder 654 and provide a cleaner overall experience for a user. The air permeable membranes 678, 680 are coupled between the support 670 and the inner walls of the channel 650, providing a seal around the absorbent material 676 and wick 656.

In some embodiments, the air permeable membranes 678, 680 could provide physical support for other components, such as the absorbent material 676, and possibly even the support 670. For example, a stainless steel mesh could provide sufficient drip resistance at least for a high viscosity vaporization substance, be air permeable, and provide mechanical or physical support.

The flaps 672, 674 are provided to control exposure of the absorbent material 676 to the channel 650. The flaps 672, 674 are semi-circular plates that have a radius approximately equal to the radius of the inner wall of the channel 650. Thus, the flaps 672, 674, in conjunction with the support 670, could be used to control air/vapor flow in the channel 650 during use of the vaporization device 600. As illustrated in FIG. 12, the flaps 672, 674 are in a position that covers the wick 656, the absorbent material 676, and the air permeable membranes 678, 680 of the holder 654. As such, air/vapor flow in the channel 650 will be diverted around these components of the holder 654. Thus, FIG. 12 illustrates an example where the holder 654 is not exposed to, or in a sense is disengaged from, the channel 650. At most, a relatively small amount of vapor produced by the atomizer 612 will flow around the flap 674 and interact with the vaporization substance from the chamber 620, and therefore this vaporization substance might be vaporized in very small quantities or not at all. If the vaporization substance from the chamber 620 is vaporized in the holder 654, at most a relatively small amount of this vapor could flow around the flap 672 and into the channel 650.

FIG. 13, on the other hand, illustrates an example of the holder 654 being exposed to, or in a sense engaged with, the channel 650. In FIG. 13, the flaps 672, 674 are in a position that does not cover the wick 656, the absorbent material 676, and the air permeable membranes 678, 680 of the holder 654. Therefore, vapor produced by the atomizer 612 is permitted to flow through the air permeable membranes 678, 680 and interact with the vaporization substance held in the wick 656 and the absorbent material 676. Thus, the vapor produced by the atomizer 612 could vaporize the vaporization substance from the chamber 620. Alternatively, the vaporization substance from the chamber 620 may simply be vaporized by the air flow created when a user inhales on the vaporization device 600.

The flaps 672, 674 could be moved from the position shown in FIG. 12 to the position shown in FIG. 13 using the user input device 630 and the switch 632. For example, when the switch 632 is in the position shown in FIG. 9, where in the intake hole 626 is closed, the flaps 672, 674 could be in the position shown in FIG. 12. This arrangement would inhibit the movement of vaporization substance from the chamber 620 into the channel 650, at the same time as disengaging the feeder 654 from the channel. This position of the switch 632 would correspond to a situation where a user does not wish to vaporize the vaporization substance in the chamber 620. When the switch 632 is in the position shown in FIG. 8, where in the intake hole 626 is open, the flaps 672, 674 could be in the position shown in FIG. 13. This arrangement would permit the movement of vaporization substance from the chamber 620 into the channel 650, at the same time as engaging the feeder 654 with the channel. This position of the switch 632 would correspond to a situation where a user wishes to vaporize the vaporization substance in the chamber 620. Alternatively, a second user input device, separate from the user input device 630, could be added to the vaporization device 600 to engage or disengage the holder 654 and the channel 650.

Other embodiments could include different types of holders. Such holders could be similar to the example shown in FIGS. 10 to 13, but include only one of the two illustrated flaps, for instance, which would still provide a level of control over exposure of the holder or vaporization substance to the channel.

In some embodiments, the base 628 includes a heater 652, inside the channel 650 and in fluid communication with the atomizer 612, to heat the vapor from the atomizer to vaporize the vaporization substance from the chamber 620. Depending on, for example, the vaporization temperature of the vaporization substance stored in the chamber 620, the heater 652 might not be implemented or desired to achieve vaporization. Therefore, the heater 652 could be omitted in some embodiments. The heater 652 is illustrated as a coil heater, however a fan heater, a ceramic heater, and/or another type of heater such as a quartz heater could also or instead be used. The user input device 630 could control power to the heater 652. For example, the heater could be turned on when the switch 632 is in the position shown in FIG. 8, and vaporization of the vaporization substance from the chamber 620 is desired. Power to the heater 652 could be provided by a battery in the battery compartment 608. In some embodiments, the base 606, the atomizer 612, the stem 610, and the base 628 could act as a conductor to provide a connection that delivers power to the heater 652 from the battery 608. However, one or more separate electrical conductors could be provided, for example, from the base 606 and along an inner or outer wall of the stem 610, along an outer or inner wall of the chamber 604, and/or elsewhere in the vaporization device 600 to deliver power to the heater 652. Conductors could be implemented using transparent conductors, such as indium tin oxide films, so that they are not noticeable to a user. Alternatively, the base 628 could include a power source, such as a battery, to power the heater 652.

A vapor heater need not necessarily be provided in a base of a secondary chamber. For example, a primary chamber could be part of a cartridge that has both a heated atomizer and an auxiliary vapor heater inside its stem. The vapor heater could be electrically connected to the cartridge base for at least power and possibly control, with the vapor heater being active or operated only if the cartridge is coupled to a secondary cartridge or chamber. Presence of a secondary cartridge could be detected by a controller, for example, and a vapor heater of a primary cartridge could be operated by the controller only if a secondary cartridge is detected.

The vaporization device 600 could also include a vapor regulator (not shown in FIG. 6 but described with reference to FIG. 5, for example) to control the flow of vapor from the atomizer 612. This vapor regulator could provide a form of dosage control.

The vaporization device 600 could allow a user to create specific mixtures of vapors produced from the vaporizations substances stored in the chambers 604, 620. These mixtures of vapors could be tailored to match a specific effect, flavor, or aromatic profile. For example, the chamber 604 could contain a vaporization substance with an active ingredient, and the chamber 620 could contain a vaporization substance with a flavorant such as a terpene. When the atomizer 612 is vaporizing the vaporization substance from the chamber 604, a user could use the user input device 630 to adjust or control the movement of the vaporization substance from the chamber 620 to the channel 650. The rate of vaporization of the vaporization substance from the chamber 620 could be determined according to any of a variety of parameters, including its vaporization temperature, properties of the feeder 654, and the temperature of the vapor from the atomizer 612 and/or the heater 652. Any or all of these parameters could be adjustable to create a vapor mixture with a desired or predefined profile.

Although only two chambers are illustrated in FIGS. 6 and 7, the chamber 620 could be one of multiple secondary chambers in fluid communication with respective feeders, which are in fluid communication with the channel 650. These multiple secondary chambers could, but need not necessarily, be stacked in a vertical or aligned arrangement. Consider a vaporization device as shown in FIGS. 3 and 4. Chambers could be interconnected such that vapor is drawn in different directions through different chambers to provide a serial configuration, even though the chambers are not physically aligned with each other.

Secondary chambers could be added or removed by a user. Additional secondary chambers, storing different flavorants for example, could allow a user to create more complex flavor profiles in a vapor mixture.

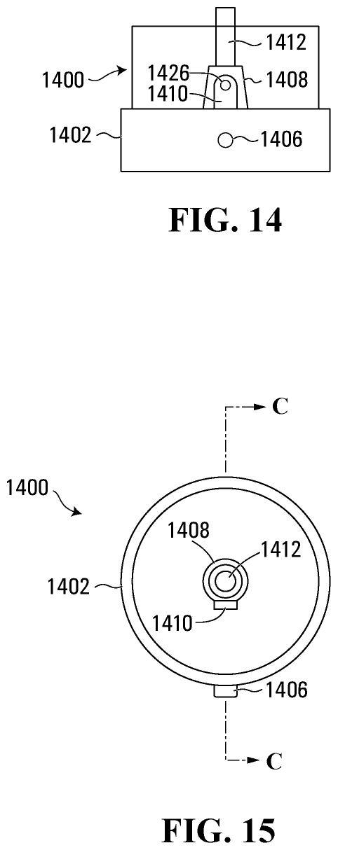

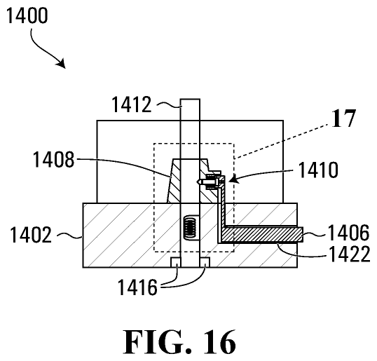

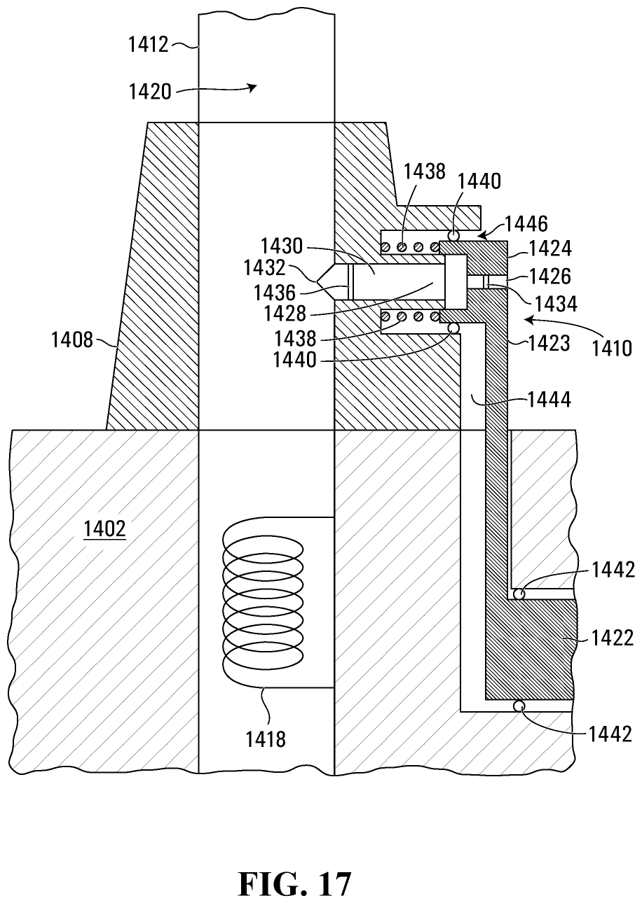

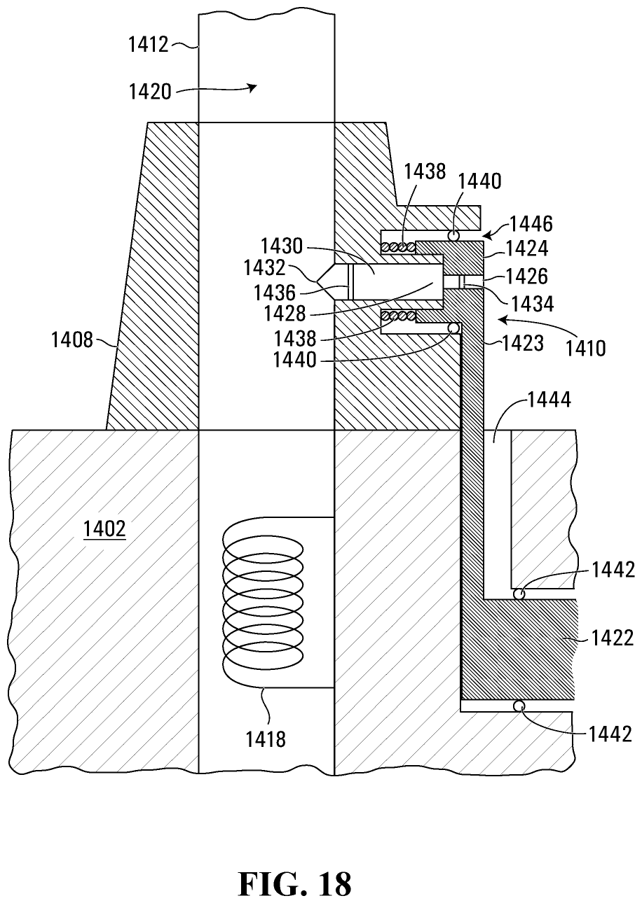

The chamber 620 illustrated in FIGS. 6-13 is one example of a secondary chamber for a vaporization device. Other examples are also contemplated. FIG. 14 is a plan view of another example secondary chamber 1400, FIG. 15 is a top view of the secondary chamber 1400, and FIG. 16 is a cross-sectional view of the secondary chamber 1400, along the line C-C in FIG. 15. FIGS. 17 and 18 are magnified portions of the cross-sectional view in FIG. 16, and provide a more detailed view of region 17 in FIG. 16.

The secondary chamber 1400 could be implemented in a multi-chamber vaporization device to store an additional vaporization substance. For example, the chamber 1400 could be implemented in the vaporization device 600 in a manner similar to the secondary chamber 620 discussed above with reference to FIGS. 6-13. In one embodiment, the chamber 1400 could be operatively coupled between the chamber 604 and the cap 602, in place of the chamber 620, to create a two-chamber vaporization device. Alternatively, the chamber 1400 could be operatively coupled between the chamber 620 and the cap 602 to create a three-chamber vaporization device. FIG. 16 illustrates notches 1416 in the base 1402, which could accommodate or engage with a stem of a primary chamber or another secondary chamber, for example. Other arrangements of the secondary chamber 1400 in a multi-chamber vaporization device are also contemplated.

The chamber 1400 is coupled to a base 1402. A button 1406 is provided on the outside surface of the base 1402. A feeder 1408, a pump 1410 and a stem 1412 are provided inside of the chamber 1400. As shown in FIG. 16, a channel 1420 is formed inside of the base 1402, the feeder 1408 and the stem 1412. The feeder 1408 engages, and could be coupled to, the base 1402, to feed the vaporization substance from the chamber 1400 to the channel 1420. The pump 1410, which is an example of a regulator to control movement of the vaporization substance from the chamber 1400 to the channel 1420, is provided in the feeder 1408. The button 1406 is an example of a user input device, which is coupled to the pump 1410, to permit or inhibit the movement of the vaporization substance from the chamber 1400 to the channel 1420. The feeder 1408, the pump 1410 and/or the button 1406, could be made of the same materials or different materials. A non-limiting list of example materials includes metals, plastics and ceramics.

The pump 1410 is provided to draw the vaporization substance from the chamber 1400 and push it through a spray nozzle 1432 to spray the vaporization substance into the channel 1420. This spray of vaporization substance could create a vapor. For example, the spray nozzle could break up the vaporization substance into small enough droplets such that they are easily carried by a flow of air/vapor moving in the channel 1420. In this regard, the feeder 1408 and/or the pump 1410 could be considered an unheated atomizer. The spray of vaporization substance could also or instead form a mist. The mist might cling to the walls of the channel 1420, where it could be vaporized. This vaporization might be stimulated using heat carried by a vapor from a primary chamber, for example. Advantageously, the relatively high surface area to volume ratio in a mist could lead to the mist being more rapidly vaporized than a steady flow of vaporization substance. The channel 1420 could include a holder, such as an absorbent material and/or air permeable membrane, to hold a mist of vaporization substance in the channel 1420. The use of a holder might provide a more consistent source vaporization substance, and/or help prevent leaks and fouling in other components of a vaporization device.

The structure of the pump 1410 is perhaps best illustrated in FIGS. 17 and 18. The pump 1410 includes a pump head 1424 in a cavity 1446 of the feeder 1408. A channel 1426, provided in the pump head 1410, fluidly connects the inside of the chamber 1400 to a pump chamber 1428. The channel 1426 includes a one-way valve 1434, to permit the vaporization substance to flow from the chamber 1400 into the pump chamber 1428, but inhibit any flow from the pump chamber 1428 back into the chamber 1400. A seal 1440, which could be a gasket or O-ring for example, is provided in the cavity 1446, between the pump head 1424 and the body of the feeder 1408, to seal the cavity and help prevent leaks around the edges of the pump head 1424. The pump chamber 1428 could hold relatively small volumes of vaporization substance. In some embodiments, the pump chamber 1428 could hold volumes in the range of 1 mL to 1 μL, however other volumes are also possible. The pump chamber 1428 is in fluid communication with a channel 1430, which is in fluid communication with the spray nozzle 1432. The channel 1430 includes another one way valve 1436, which permits the flow of vaporization substance from the pump chamber 1428 into the channel 1420, but inhibits flow from the channel into the pump chamber.

The pump head 1424 is rigidly coupled to a vertical member 1423, which is coupled to a horizontal member 1422 in the example shown. In other embodiments an in-line shaft could extend from the pump head 1424 through a seal or other sealing structure in a chamber wall.

A cavity 1444 in the base 1402 accommodates the vertical member 1423 and the horizontal member 1422. A seal 1442, such as a gasket or O-ring, is provided in the cavity 1444, between the horizontal member 1422 and the base 1402, to seal the cavity and help prevent leaks from the chamber 1400. A resilient member 1438, such as a spring, biases the pump head 1424 into the position illustrated in FIG. 17. The inner walls of the cavity 1444 confine the motion of the vertical member 1423 and the horizontal member 1422, and therefore the pump head 1424, to a desired range. The button 1406 is provided on the distal end of the horizontal member 1422, relative to the vertical member 1423. The button 1406 is part of a shaft, specifically the horizontal member 1422, in the example shown. In other embodiments a button could be implemented as a separate component. A pump could also or instead be driven otherwise than by a mechanical linkage. For example, a user input device or controller could have a wired or wireless connection to a drive component or element that is capable of moving the pump head 1424 or otherwise capable of operating a pump.

In the illustrated embodiment, a user may push the button 1406 to overcome the force of the resilient member 1438, and move the pump 1410 into the configuration or state shown in FIG. 18. In this configuration, the volume available for the vaporization substance in the pump chamber 1428 is reduced. Therefore, the vaporization substance present in the pump chamber 1428 becomes pressurized. The vaporization substance cannot flow back into the chamber 1400 due to the action of the one-way valve 1434 and the gasket 1440, therefore the vaporization substance is forced through the channel 1430, the one-way valve 1436 and the spray nozzle 1432, into the channel 1420. This creates a mist or vapor of the vaporization sub stance.

When the user releases the button 1406, the resilient member 1438 biases the pump 1410 back into the configuration shown in FIG. 17. As this time, the volume of the pump chamber 1428 increases, which decreases the pressure in the pump chamber. Due to the one-way valve 1436, air/vapor cannot flow from the channel 1420 to the pump chamber 1428. Therefore, vaporization substance could be drawn from the chamber 1400, through the channel 1426 and the one-way valve 1434, and into the pump chamber 1428. In a sense, this movement of vaporization substance from the chamber 1400 to the pump chamber 1428 primes the pump 1410 for the next time a user presses the button 1406.

As shown in FIG. 16, the base 1402 could include a heater 1418, inside the channel 1420, to heat air/vapor entering the channel 1420 and vaporize the vaporization substance from the chamber 1400. The heater 1418 could be similar to the heater 652 discussed above. Depending on, for example, the vaporization temperature of the vaporization substance stored in the chamber 1400, the heater 1418 might not be necessary or desired to achieve vaporization. Therefore, the heater 1418 could be omitted, or provided and not powered or operated, in some embodiments. The heater 1418 is illustrated as a coil heater, however other types of heater such as a fan heater, a ceramic heater, and/or a quartz heater could also or instead be used. The button 1406 could control power to the heater 1418 such that the heater is turned on while and shortly after the button is pressed. This could provide the necessary heat in channel 1420 to better vaporize the vaporization substance from the chamber 1400. Power could be supplied to the heater 1418 from a battery in a vaporization device. Alternatively, the base 1402 could include a power source, such as a battery, to power the heater 1418.

FIGS. 14-18 illustrate one embodiment to create a spray or mist of vaporization substance in a channel. However, other embodiments are also contemplated. For example, a secondary chamber storing a vaporization substance could be pressurized. The chamber could be in fluid communication with a channel through a valve and a spray nozzle. When a user wishes to spray the vaporization substance into the channel, the valve could be opened and pressure in the chamber could force the vaporization substance through the spray nozzle. In this example, the pressure in the chamber could reduce or eliminate a need for a pump.

In some embodiments, a secondary chamber with a pump and spray nozzle could be operatively coupled upstream of a primary chamber in a multi-chamber vaporization device. In these embodiments, the secondary chamber could use a spray nozzle to create a mist or vapor of vaporization substance that flows into an atomizer coupled to the primary chamber, for example. The atomizer could produce another vapor from a vaporization substance in the primary chamber, which is mixed with the vapor from the spray nozzle of the secondary chamber. The atomizer could also help to vaporize any un-vaporized substance produced from the spray nozzle.

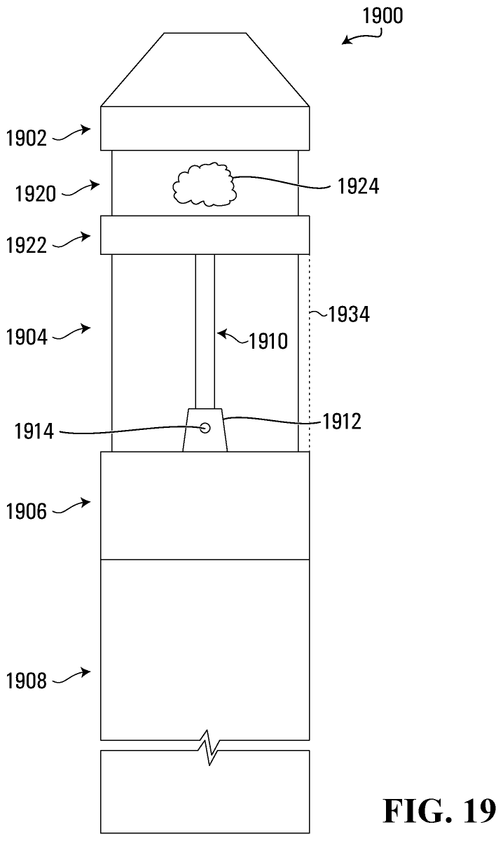

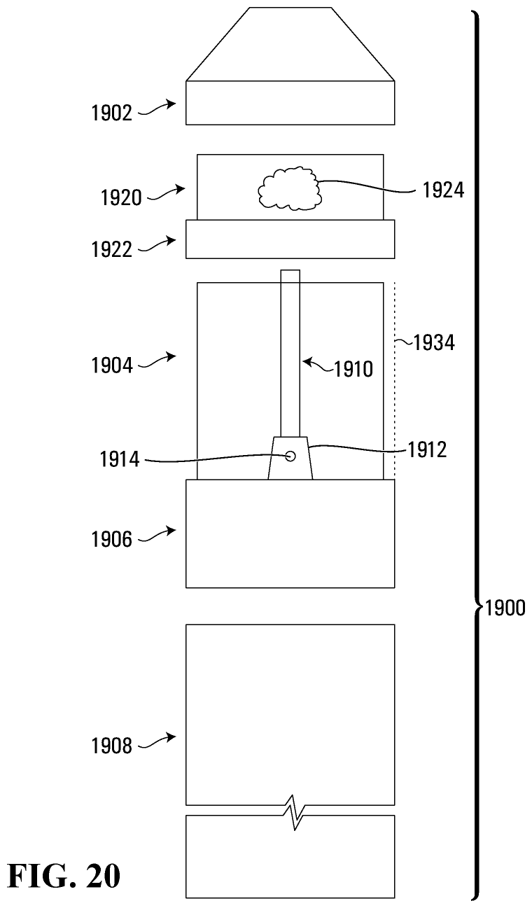

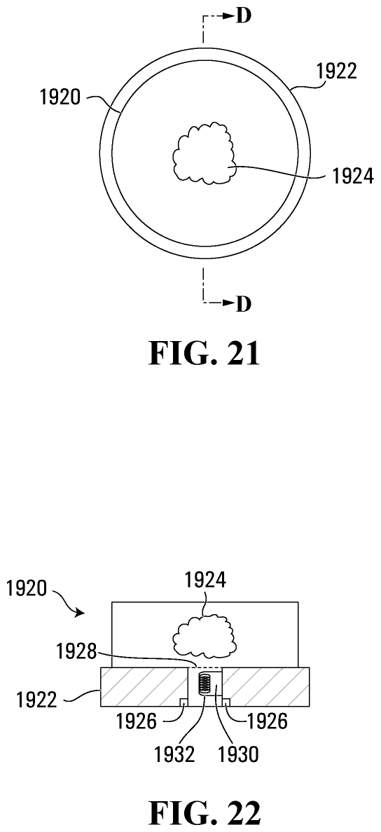

FIGS. 6 to 18 illustrate example secondary chambers that include feeders. However, secondary chambers without feeders are also contemplated. FIG. 19 is a plan view of another example vaporization device 1900 that includes a secondary chamber 1920 without a feeder. FIG. 20 is a plan and partially exploded view of the vaporization device 1900, FIG. 21 is a top view of the secondary chamber 1920, and FIG. 22 is a cross-sectional view of the secondary chamber 1920, along the line D-D in FIG. 21.

The vaporization device 1900 includes, in part, a cap 1902, primary chamber 1904, a base 1906, a battery compartment 1908, a stem 1910, an atomizer 1912, and an intake hole 1914. These components could be similar to the cap 102, chamber 104, base 106, battery compartment 108, stem 110, atomizer 130 and intake hole 134, respectively, which are discussed above with reference to FIGS. 1 and 2.

The chambers 1904, 1920 could be recloseable or non-recloseable, and could be of the same size, or could have different sizes. FIG. 22 illustrates notches 1926 in the base 1922, which could accommodate or engage the distal end of the stem 1910 relative to the atomizer 1912. The primary chamber 1904 stores a vaporization substance, and is in fluid communication with the atomizer 1912 through the intake hole 1914. The atomizer 1912 generates a vapor from this vaporization substance by, for example, heating the vaporization substance. The stem 1910 contains a channel in fluid communication with the atomizer 1912. Vapor produced in the atomizer 1914 flows through this channel. The channel is in fluid communication with another channel 1930, which is provided in the base 1922. The channel 1930 is in fluid communication with the inside of secondary chamber 1920.

The secondary chamber 1920 stores a vaporization substance 1924. The vaporization substance 1924 is illustrated as a solid, however other types and forms of vaporization substance are possible. The secondary chamber 1920 could include a carrier such as an absorbent material carrying a liquid vaporization substance. A user could reload and/or replace carriers in a recloseable cartridge or chamber, for example. Multiple carriers be placed into a chamber. Carriers could be stacked or otherwise arranged so that air is drawn through a combination of multiple vaporization substances, or a chamber could be divided into multiple compartments that can be selectively, individually or in combination, exposed to the channel 1930.

In the example shown, the vaporization substance 1924 is contained within the chamber 1920 by the walls of the chamber, the cap 1902, and the base 1922. An air permeable membrane 1928, provided between the inside of chamber 1920 and the channel 1930, could help prevent the vaporization substance 1924 from entering the channel. The air permeable membrane 1928 could be made from any of various air permeable materials suitable to permit air flow while inhibiting entry of the vaporization substance 1924 into the channel 1930.

A heater 1932 is inside the channel 1930, and is in fluid communication with the atomizer 1912. This heater 1932 could be similar to the heater 652 , and/or the heater 1418 discussed above. The heater 1932 could heat vapor produced by the atomizer 1912, or it could heat air inside of the channel 1930. The vaporization substance 1924 could then be vaporized by the vapor and/or air that is heated by the heater 1932.

In some embodiments, the vaporization substance 1924 is a flower containing cannabinoids. The vaporization temperature of some cannabinoids is relatively high, and therefore the temperature of the vapor produced by the atomizer 1912 might be insufficient to vaporize the vaporization substance 1924. In these embodiments, the heater 1932 could be used to increase the vapor/air temperature in the channel 1930 to a temperature equal to or greater than the vaporization temperature of the vaporization substance 1924.

Heating of the vaporization substance 1924 could also be a relatively slow process. For example, the time required to heat the vaporization substance 1924 to its vaporization temperature could be longer than the time it takes for a user to inhale from the vaporization device 1900. A regulator, channel, and/or other device could be implemented in the chamber 1920 to circulate the flow of heated vapor/air around the vaporization substance 1924 and/or provide more time for the vaporization substance to heat up to its vaporization temperature, and could be useful for a vaporization substance having a higher vaporization temperature or heating time.

The vaporization device 1900 includes an electrical connection 1934 to deliver or transfer power from the base 1906 to the base 1922. Other powering and control connections or arrangements are also disclosed elsewhere herein.

Power to the base 1906 could originate from a battery in the battery compartment 1908. The power delivered or transferred to the base 1922 could be used by the heater 1932.

This power could be controlled based on, for example, the vaporization temperature of the vaporization substance 1924 and/or the temperature of the vapor/air entering the channel 1930 from the atomizer 1912. A sensor (not shown) in the channel 1930 or the stem 1910 could be used to determine the temperature of the vapor/air entering the channel 1930. The power delivered to the heater 1932 could also or instead be controlled based on the flow rate of the air/vapor in the channel 1930. When there is a high vapor/air flow rate, i.e. when a user is inhaling heavily, the power delivered to the heater 1932 could be increased accordingly. Another sensor could be used in the channel 1930 or the stem 1910 to determine the flow rate of the vapor/air.

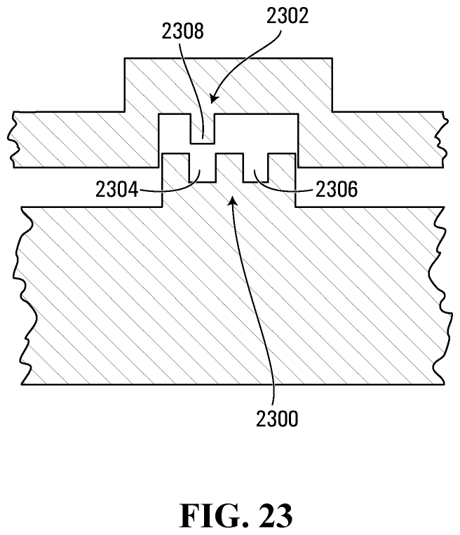

Control of the power delivered to the heater 1932 could also or instead be determined and/or controlled in the base 1906 and/or the battery compartment 1908. In embodiments where the chamber 1920 is replaceable, the chamber 1920 and/or the base 1922 could include an indicator of the vaporization substance 1924, and the power to the heater 1932 could be control based on that indicator. An example of indicators that could be used with the chamber 1920 and/or the base 1922 is provided in FIG. 23, which is discussed in detail below.

Secondary chambers with and without feeders are disclosed by way of example herein. In the particular examples shown in FIGS. 6 to 22, a vaporization substance in a secondary chamber is fed directly into a channel through which the vapor produced from the vaporization substance in a primary chamber also flows. It should be appreciated that this is not the only way to provide for feeding or introduction of a secondary vapor or vaporization substance into a channel. A feeder of a secondary chamber could be at least partially located in a separate channel that is in fluid communication with a primary channel, for example. This could still be considered a form of “pull-through” vaporization or a serial configuration in that the vapors produced from multiple vaporization substances are added to a channel sequentially, although one or more secondary vapors or vaporization substances are fed or introduced into the channel indirectly, through one or more separate channels. An illustrative example is shown in FIGS. 22A to 22E.

FIGS. 22A and 22B are plan views of an example secondary chamber, FIG. 22C is a top view of the secondary chamber in FIG. 22A, FIG. 22D is a cross-sectional view of the secondary chamber in FIG. 22A along the line E-E in FIG. 22C, and FIG. 22E is a plan view of a mouthpiece that could be used with the secondary chamber in FIG. 22A.

The secondary chamber 2200 could be implemented in a multi-chamber vaporization device. For example, the chamber 2200 could be implemented in the vaporization device 600 in a manner similar to the secondary chamber 620 discussed above with reference to FIGS. 6-13. In one embodiment, the chamber 2200 could be operatively coupled between the chamber 604 and the cap 602, in place of the chamber 620, to create a two-chamber vaporization device. Alternatively, the chamber 2200 could be operatively coupled between the chamber 620 and the cap 602 to create a three-chamber vaporization device. FIG. 22D illustrates notches 2240 in the base 2230, which could accommodate or engage with a stem of a primary chamber or another secondary chamber, for example. Other arrangements of the secondary chamber 2200 in a multi-chamber vaporization device are also contemplated.

The chamber 2200 is coupled to a base 2230. Tabs 2232, 2234 are provided on the outside surface of the base 1402. A feeder 1408, a pump 1410 and a stem 1412 are provided to operate doors or covers, one of which is shown by way of example in FIG. 22B, to control air intake into stems 2202, 2206. As shown in FIG. 22D, a pass-through channel is formed at 2244 inside of the base 2230 and through the stem 2204. The pass-through channel is not in fluid communication with the chamber 2200. The stems 2202, 2206 include feeders in the form of intake holes 2222, 2224, 2226, 2228, 2229. In an embodiment, each of the stems 2202, 2206 includes four intake holes, at 90 degree intervals around the periphery of each stem. More generally, each of the stems 2202, 2206 could be perforated and include one or more perforations as a form of feeder to feed a vaporization substance from the secondary chamber 2200 into a primary channel indirectly, through a separate secondary chamber channel. In the example shown, there are multiple separate secondary channels provided in part by the stems 2202, 2206.

The intake holes 2222, 2224, 2226, 2228, 2229 are an example of one form of feeder. Other forms of feeder, including the other examples disclosed herein, could also or instead be provided in conjunction with separate secondary chamber channels.

The stems 2202, 2204, 2206 engage, and could be coupled to, the base 2230. The tube 2204 is part of a pass-through channel through the secondary chamber 2200, but is isolated from the secondary chamber in the sense that a vaporization substance in the secondary chamber 2200, and any vapor generated from that substance, is not fed or introduced directly into that channel. The channel through the tube 2204 is also isolated from the separate secondary chamber channels through the stems 2202, 2206 in the sense that vapor that is generated from the vaporization substance(s) in any upstream chambers does not flow through the separate secondary chamber channels.

Such separation or isolation of secondary chamber channels could be useful in helping prevent cross-contamination of secondary vaporization substances or vapors, such as flavorant vapor, with an vaporization substance in another chamber or a vapor generated therefrom, such as an active vaporization substance or active vapor. This could, for example, help avoid having vaporized cannabis resin deposit on and clogging the feeder for a secondary chamber. A secondary chamber could have its own passive or active airway(s) to feed into a mouthpiece or other channel independently of other vapor, such as a cannabis resin vapor for example.

The separate secondary chamber channels are shown perhaps most clearly in FIG. 22D, and include channels 2242, 2246 in the base 2230 and channels through the stems 2202, 2206. This is one example implementation, and separate secondary chamber channels could include channels that extend through chamber walls instead of or in addition to the base 2230.

In the example shown, the secondary chamber channels through the stems 2202, 2206 are passive channels. A channel, whether a secondary chamber channel or another channel, could be active or passive. Air is passively drawn into a passive channel when a user inhales on a mouthpiece that is in fluid communication with the channel. An active channel, whether in a secondary chamber or elsewhere, has air forced through the channel with a fan and/or other driven component.

The tabs 2232, 2234 represent an example of user input devices to control intake of air into the separate secondary chamber channels through the stems 2202, 2206. With reference to FIG. 2B, a door 2236 could be slidable within a window or aperture 2238 and a cavity in the base 2230, to control how much of the channel 2242 is unobstructed for air intake. Air intake control could be implemented in a similar manner for the channel 2246.

The tabs 2232, 2234 and slidable doors such as 2236 represent one example of air intake control for separate secondary chamber channels. Other types of air intake control are also contemplated. An apertured ring or cylinder could be rotatably mounted on or in the base 2230 to enable a user to position one or more apertures relative to air intake ends of any secondary chamber channels to thereby control air intake for multiple separate channels with one user input device. Air intake could also or instead be controlled using one or more manually operated or controlled valves, at an intake end of a separate secondary chamber channel, at an outlet of a separate channel, and/or within a separate channel, for example. A door or intake cover or plug need not be slidable, and could be movable away from the base 2230 and/or into and out of the intake end of a secondary chamber channel. The intake end of a separate secondary chamber channel could even be open, and a user could partially or fully block the intake end to control air intake.

The secondary chamber 2200 provide three channels, through the stems 2202, 2204, 2206, and could be used with a multi-channel mouthpiece, an example of which is shown in FIG. 22E. The mouthpiece 2250 could be made from any of various materials, such as those disclosed elsewhere herein by way of example with reference to caps, covers, and/or mouthpieces. Examples of how a cap, cover, or mouthpiece could engage or be coupled to a chamber are also disclosed elsewhere herein.

In the embodiment shown, the mouthpiece 2250 includes an outlet channel 2252, through which a user inhales. A central channel 2256 and two separate channels 2254, 2258 are in fluid communication with the outlet channel 2252. When engaged with the secondary chamber 2230, the mouthpiece channels 2254, 2256, 2258 are also in fluid communication with the secondary chamber channels through the stems 2202, 2204, 2206, respectively. Such an arrangement could provide for cannabis vapor flow-through with no vapor mixing until vapor flows reach the outlet channel 2252, with isolated air paths for flavorant vapor and flavorant vapor mixing with cannabis vapor only in the mouthpiece 2250, for example. This could reduce or avoid clogging of upstream components such as secondary chamber feeders by cannabis resin vapor depositing on those components.

FIGS. 22A to 22E represent examples of a secondary chamber with separate channels and a mouthpiece that could be used in conjunction with such a secondary chamber.

Other embodiments could be similar or different. For example, a secondary chamber could have more or fewer than two separate secondary chamber channels. The stems through which different channels are provided need not necessarily be in-line in cross section as shown in FIG. 22C, and pass-through and separate channels could be arranged otherwise than with a central pass-through channel.

Other features disclosed elsewhere herein could also or instead be implemented in a secondary chamber with separate channels.

Several embodiments herein reference chamber engagement structures. FIG. 23 is a cross-sectional and partially exploded view of an example of engagement structures in a vaporization device. FIG. 23 illustrates an engagement structure 2300 and a complementary engagement structure 2302. Engagement structures could be used with replaceable or reconfigurable secondary chambers in a vaporization device. These engagement structures could be useful for restricting a vaporization device to a particular model or type of secondary chamber. Engagement structures could also or instead be useful as an assembly aid, to ensure that secondary chambers are assembled properly. Further, the engagement structure for a secondary chamber could include an indicator of the vaporization substance stored in the chamber. A vaporization device could read this indicator to determine the type of vaporization substance in the secondary chamber. For example, some chambers or cartridges may include one or more active coolers, and a vaporization device may adapt power supply and/or control to a chamber or cartridge according to chamber or cartridge type.

In some embodiments, the engagement structure 2302 could be provided on the base of a secondary chamber, at the point where the base contacts and/or engages with a primary chamber. Engagement structure 2300 could be provided on the primary chamber, at the point where the primary chamber contacts and/or engages with the base. In a specific example, referring to FIG. 6, the engagement structure 2302 could be provided on the base 628 near the engagement 634, and the engagement structure 2300 could be provided on the primary chamber 604, also near the engagement 634. When the base 628 and the primary chamber 604 are engaged via the engagement 634, the engagement structures 2300, 2302 are also engaged. However, in general, other implementations are possible, such as providing engagement structures on stems, caps and/or other components of a vaporization device.

In the embodiment illustrated in FIG. 23, the engagement structure 2300 is sized to engage with the complementary engagement structure 2302. Therefore, only components with structures similar to the engagement structure 2300 will be able to couple to components containing the engagement structure 2300.

The engagement structure 2300 includes notches 2304 and 2306, and the complementary engagement structure 2302 includes a protrusion 2308. The protrusion 2308 could include a conductive pin and the notches 2304 and 2306 could include contacts, for example, to provide for detection of an installed chamber or cartridge and/or an installed chamber or cartridge type. Other embodiments are also contemplated, and the notches 2304 and 2306 could include pressure sensors or another type of sensor to detect the presence of a protrusion 2308.

Engagement structures that are similar to or different from the examples shown in FIG. 23 could be more specific to particular types of chambers. One or more engagement structures on an apparatus such as a vaporization device could mechanically restrict chambers, cartridges, and/or other components to only specific types. An engagement structure could include one or more features, such as one or more protrusions and/or one or more grooves, with size(s), shape(s), and/or positions to mate only with a particular type of cooperating component with one or more complementary features. This type of physical or mechanical specificity could be used, for example, to restrict a vaporization device to use with only certain types of chambers or cartridges, which could provide a measure of control over the particular vaporization substances that are available for vaporization by a vaporization device. Certain chambers or cartridges could be restricted to certain positions, which could have regulators, power supply terminals, and/or other features that are specially adapted for those chambers or cartridges, for example.

As noted above, engagement structures need not have only a physical function such as controlling correct placement or alignment of a chamber and/or other component or limiting chambers and/or other components to particular types. Engagement structures on different chambers could have different sizes and/or patterns of conductive pins, for example, to enable a vaporization device to detect the type(s) of chambers that have been installed.

In the example of FIG. 23, the presence of the protrusion 2308 aligned with the notch 2304 and the lack of a protrusion aligned with the notch 2306 could provide information regarding an installed chamber. This information could include the type of vaporization substance stored by a chamber, which could be used by a controller, in a base of a multi-chamber cartridge or elsewhere in a multi-chamber device, for example, to control the voltage, current, and/or power supplied to an atomizer or to a heater. One or more regulators and/or feeders within a multi-chamber cartridge or device could also or instead be controlled based on the type of vaporization substance stored by the chamber.

Each different type of chamber that is compatible with a multi-chamber cartridge or device could have a unique engagement structure. The two notches 2304 and 2306 in FIG. 23 can detect a maximum of four different types of chambers, including chambers with no protrusions, chambers with two protrusions, chambers with only one protrusion 2308 as shown, and chambers with only one protrusion that corresponds to notch 2306. However, engagement structures with more or fewer notches could be used to different numbers of chamber types.

The protrusions and notches illustrated in FIG. 23 are provided by way of example only. Other arrangements, sizes, and shapes of engagement structures that might or might not include protrusions and/or grooves are also contemplated. Although described above primarily in the context of chambers, engagement structures could also or instead be used in conjunction with cartridges and/or other components. Engagement structures are also not in any way limited to localized structures at certain locations on or in an apparatus or component. Different types of chamber or cartridge could have different shapes that will only fit into compartments, such as those shown at 313 in FIG. 4, for example, that have a complementary shape.

Embodiments described above relate primarily to multi-chamber apparatus such as vaporization devices. Other embodiments, including methods, are also contemplated.

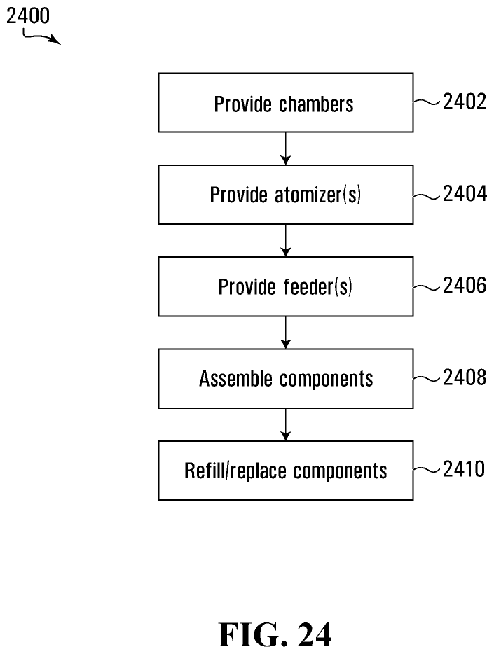

FIG. 24, for example, is a flow diagram illustrating a method 2400 according to an embodiment. The example method 2400 involves an operation 2402 of providing chambers to store vaporization substances, an operation 2404 of providing one or more atomizers to generate vapor from a vaporization substance by heating the vaporization substance, and operation 2406 of providing one or more feeders to feed a vaporization substance from a chamber to a channel.

These operations 2402, 2404 and 2406 are shown separately for illustrative purposes, but need not be separate operations in all embodiments. For example, a vaporization device could include a feeder and an atomizer, and could also be sold with vaporization substance chambers as well. A vaporization device that is usable with multiple chambers, or components thereof, could potentially be provided separately from the chambers, which could be purchased separately, for example, and therefore the operation of providing chambers is optional in at least some embodiments. Some chambers could be provided with a vaporization device, while others could be sold separately. For example, a primary chamber could be provided with a vaporization device, while one or more secondary chambers could be sold separately.

The chambers, atomizer(s) and/or feeder(s) could be provided at 2402, 2404, 2406 by actually manufacturing these components. Any of these components, and/or other components, could instead be provided by purchasing or otherwise acquiring the components from one or more suppliers.

At least some components or parts thereof could be provided in different ways. Different cartridge parts, such as chambers, bases, caps, and atomizers, for example, could be provided by manufacturing one or more parts and purchasing one or more other parts, or by purchasing different parts from different suppliers.

Providing chambers at 2402 could include providing at least a primary chamber and a secondary chamber. The operation 2402 could also include providing at least one further secondary chamber.

Providing a feeder at 2406 could include providing a regulator to control movement of a vaporization substance from a secondary chamber to a channel. The regulator could, for example, include a spray nozzle to spray the vaporization substance into the channel or a separate channel. The method 2400 could also include providing a user input device to control the regulator to permit or inhibit the movement of the vaporization substance from the secondary chamber to the channel. The operation 2406 could further include providing a holder to hold a vaporization substance in a channel or a separate channel, and the method 2400 could further include providing a user input device to control exposure of the holder to the channel or a separate channel. The user input device to control the regulator could be the same as or different from the user input device used to control exposure of the holder to the channel or a separate channel.

In some embodiments, components such as the atomizer(s) provided at 2404 and the feeder(s) provided at 2406, and possibly the chambers provided at 2402, are provided in the form of a pre-assembled vaporization device. In other embodiments, components are not necessarily assembled. FIG. 24 therefore also illustrates an operation 2408 of assembling components. This could involve, for example, arranging an atomizer in fluid communication with a chamber and/or a channel, such as by installing the atomizer, the channel and/or the chamber in a vaporization device or cartridge. The operation 2408 could further involve arranging a feeder in fluid communication with a channel and/or a chamber, and possibly arranging the feeder downstream of the atomizer. Moreover, the operation 2408 could involve arranging at least a portion of the feeder inside of a channel.

Providing the chambers at 2402 could involve providing a chamber with an engagement structure to engage with a complementary engagement structure of a vaporization device, in which case assembly at 2408 could involve arranging the chamber with the engagement structure engaging with the complementary engagement structure of the vaporization device. Further, providing the chamber at 2402 could involve providing a chamber including an indicator of a vaporization substance.

One or more components, such as chambers, could be refilled or replaced as shown at 2410.

The example method 2400 is illustrative of one embodiment. Examples of various ways to perform the illustrated operations, additional operations that may be performed in some embodiments, or operations that could be omitted in some embodiments, could be inferred or apparent from the description and drawings, for example. Further variations may be or become apparent. Not all embodiments necessarily involve all of the operations shown in FIG. 24. For example, not all embodiments employ feeders.

In some embodiments, the method 2400 could further include steps of providing and/or arranging a channel in fluid communication with an atomizer, providing and/or arranging a mouthpiece in fluid communication with the channel, providing and/or arranging a vapor regulator to control a flow of the vapor from an atomizer, and/or providing and/or arranging a heater, in fluid communication with an atomizer, to heat the vapor from the atomizer to vaporize the second vaporization substance. A cooler could also or instead be provided and/or arranged in a vaporization device. The channel, the mouthpiece, the vapor regulator, the heater and/or the cooler could be provided with other components, such as a chamber, an atomizer and/or a feeder, or separately from other components.

Other features disclosed herein could also apply to method embodiments. For example, vaporization substances could be or include at least one of: a liquid, a gel and a wax. Providing chambers at 2402 could include providing a first chamber storing a vaporization substance including an active substance, and/or providing a second chamber storing a vaporization substance including a flavorant. The active substance could include a cannabinoid, and the flavorant could include any one or more of: a terpene, an essential oil, and a volatile plant extract. The flavorant could be used to control an effect, flavor and/or aromatic profile of the vaporization substance containing the active substance during vaporization. This effect, flavor and/or aromatic profile could be tuned in a multi-chamber vaporization device to suit a user's preference.

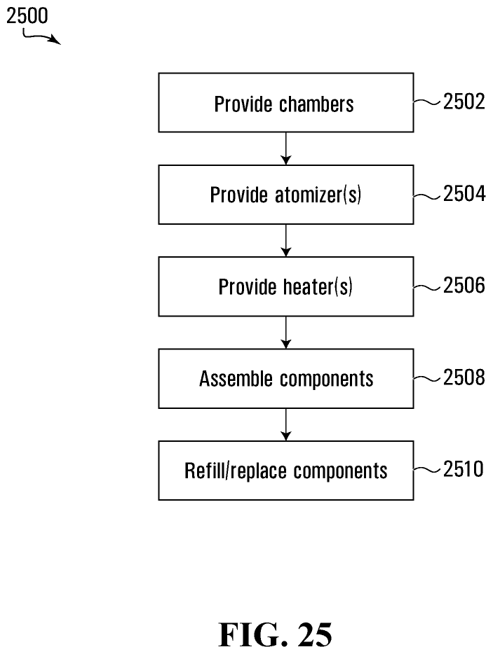

FIG. 25 is another flow diagram illustrating a method 2500 according to an embodiment. The example method 2500 involves an operation 2502 of providing chambers to store vaporization substances, an operation 2504 of providing one or more atomizers to generate vapor from a vaporization substance by heating the vaporization substance, and operation 2506 of providing one or more heaters to heat vapor from an atomizer. The example method 2500 also includes an operation 2508 of assembling components, and an operation of 2510 of refilling and/or replacing one or more components. The operations 2502, 2504, 2508, 2510 could be similar to the operations 2402, 2404, 2408, 2410 discussed in detail above with reference to FIG. 24.

The operation 2508 could involve, for example, arranging an atomizer in fluid communication with a chamber and/or a channel, such as by installing the atomizer, the channel and/or the chamber in a vaporization device or cartridge. The operation 2508 could further involve arranging a heater in fluid communication with an atomizer and/or a chamber, which could store a vaporization substance for vaporization by vapor that is heated by the heater. Moreover, the operation 2508 could involve arranging at least a portion of the heater inside of a channel.

In some embodiments, the method 2500 could further include the steps of providing and/or arranging a channel in fluid communication with an atomizer and a chamber, providing and/or arranging a mouthpiece in fluid communication with the channel, and/or providing and/or arranging a vapor regulator to control a flow of vapor from an atomizer to a heater. A cooler could also or instead be provided and/or arranged in a vaporization device. The method 2500 could also or instead include providing a regulator to control movement of a vaporization substance from a chamber to a channel, and/or providing a user input device to control the regulator to permit or inhibit the movement of the vaporization substance from the chamber to the channel. The method 2500 could further include providing a holder to hold a vaporization substance in a channel or a separate secondary chamber channel, and/or providing a user input device to control exposure of the holder to the channel or a separate channel. The user input device to control the regulator could be the same as or different from the user input device used to control exposure of the holder to the channel or a separate channel. The channel, the mouthpiece, the vapor regulator, the regulator and/or the holder could be provided with other components, such as a chamber, an atomizer, a heater and/or a cooler, or separately from other components.

The example method 2500, like the example method 2400, is an illustrative and non-limiting example. Various ways to perform the illustrated operations, additional operations that may be performed in some embodiments, or operations that could be omitted in some embodiments, could be inferred or apparent from the description and drawing or otherwise be or become apparent. Other variations of methods associated with manufacturing or otherwise producing a multi-chamber apparatus such as a cartridge or a vaporization device may be or become apparent.

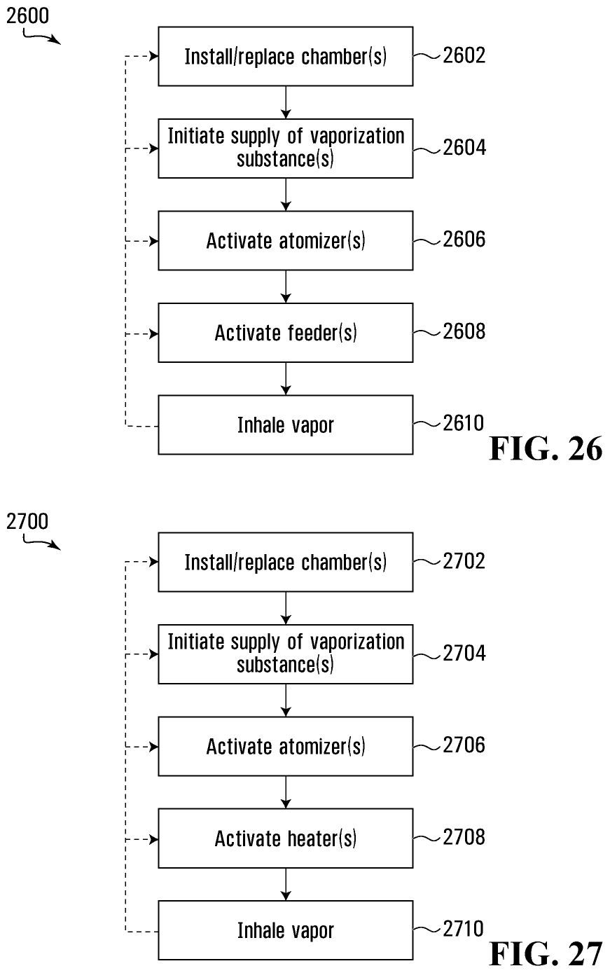

User methods are also contemplated. FIGS. 26 and 27 are flow diagrams illustrating methods 2600 and 2700 according to embodiments.

The example method 2600 involves an optional operation 2602 of installing or replacing one or more chambers. A user need not necessarily install or replace chambers every time a vaporization substance mixture is to be vaporized. The example method 2600 also involves an operation 2604 of initiating supply of one or more vaporization substances to one or more atomizers, an operation 2606 of activating the one or more atomizers, and an operation 2608 of activating one or more feeders. These operations could involve operating one or more input devices such as a control button or switch or even just inhaling on a mouthpiece. The operations at 2604, 2606, 2408 are shown separately in FIG. 26 solely for illustrative purposes, and need not necessarily be separate operations.

Similarly, inhaling vapor is shown separately at 2610, but in some embodiments inhaling on a mouthpiece initiates vaporization substance flow and vaporization.

The example method 2700 in FIG. 27 involves an optional operation 2702 of installing or replacing one or more chambers, an operation 2704 of initiating supply of one or more vaporization substances, an operation 2706 of activating one or more atomizers, and an operation 2710 of inhaling vapor. These operations could be similar to the operations 2602, 2604, 2606, 2610 of FIG. 24. The example method 2700 also includes an operation 2708 of activating one or more heaters. Similar to feeders, heaters may be activated by, for example, operating user input devices or inhaling on a mouthpiece.

The dashed arrows in FIG. 26 and FIG. 27 illustrate that multiple doses of a vaporization substance or mixture could be vaporized, and that available vaporization substances could be changed by installing or replacing one or more chambers.

In some embodiments, initiating vaporization of a first vaporization substance to produce a first vapor could be performed at 2604 and/or 2606. Initiating feeding of a second vaporization substance into a channel to produce a second vapor could be performed at 2608. Inhaling the first vapor and the second vapor could then be performed at 2610.

In other embodiments, initiating vaporization of a first vaporization substance to produce a first vapor could be performed at 2704 and/or 2706. Initiating heating of the first vapor, and initiating vaporization of the second vaporization substance by the first vapor that is heated by the heater to produce a second vapor could be performed at 2708. Vaporization of the second vaporization substance could be initiated by, for example, feeding the vaporization substance into a channel. Inhaling the first vapor and the second vapor could then be performed at 2710.

The example methods 2600, 2700 are illustrative and non-limiting examples. Various ways to perform the illustrated operations, additional operations that may be performed in some embodiments, or operations that could be omitted in some embodiments, could be inferred or apparent from the description and drawing or otherwise be or become apparent.

It should be appreciated that the drawings and description herein are intended solely for illustrative purposes, and that the present invention is in no way limited to the particular example embodiments explicitly shown in the drawings and described herein.

What has been described is merely illustrative of the application of principles of embodiments of the present disclosure. Other arrangements and methods can be implemented by those skilled in the art.

Illustrative embodiments have been described with reference to specific features and examples, various modifications and combinations can be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although embodiments and potential advantages have been described by way of example in detail, various changes, substitutions and alterations can be made herein without departing from the invention as defined by the appended claims. Moreover, the scope of the present application is not intended to be limited to the particular embodiments of any process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

1. An apparatus comprising:
a first chamber to store a first vaporization substance;
an atomizer, in fluid communication with the first chamber, to generate vapor from the first vaporization substance by heating the first vaporization substance;
a channel, in fluid communication with the atomizer;
a second chamber to store a second vaporization substance;
a feeder, in fluid communication with the channel and the second chamber, to feed the second vaporization substance from the second chamber to the channel.

2. The apparatus of claim 1, wherein the feeder is downstream from the atomizer, wherein the second vaporization substance is vaporized by heat from the vapor.

3. (canceled)

4. The apparatus of claim 1, wherein the feeder comprises an unheated atomizer.

5. (canceled)

6. The apparatus of claim 1, wherein at least a portion of the feeder is inside the channel.

7. The apparatus of claim 1, wherein at least a portion of the feeder is inside a separate channel that is in fluid communication with the channel.

8. The apparatus of claim 1, wherein the feeder comprises a regulator to control movement of the second vaporization substance from the second chamber to the channel,
wherein the regulator comprises any one or more of: a wick, a valve, a pump, a spray nozzle to spray the second vaporization substance, a mechanical feed structure, and a screw conveyor.

9. The apparatus of claim 1, wherein the feeder comprises a regulator to control movement of the second vaporization substance from the second chamber to the channel, the apparatus further comprising:
a user input device to control the regulator to permit or inhibit the movement of the second vaporization substance from the second chamber to the channel.

10-15. (canceled)

16. The apparatus of claim 1, wherein the feeder comprises a holder to hold the second vaporization substance in the channel,
wherein the holder comprises any one or more of: a wick, an absorbent material, and an air permeable material.

17. The apparatus of claim 1, wherein the feeder comprises a holder to hold the second vaporization substance in the channel, the apparatus further comprising:
a user input device to control exposure of the holder to the channel.

18-28. (canceled)

29. The apparatus of claim 1, wherein the second chamber is one of a plurality of chambers in fluid communication with respective feeders that are in fluid communication with the channel.

30. The apparatus of claim 1, further comprising:
a heater, in fluid communication with the atomizer, to heat the vapor from the atomizer to vaporize the second vaporization substance.

31. An apparatus comprising:
a first chamber to store a first vaporization substance;
an atomizer, in fluid communication with the first chamber, to generate vapor from the first vaporization substance by heating the first vaporization substance;
a heater, in fluid communication with the atomizer, to heat the vapor from the atomizer; and
a second chamber, in fluid communication with the heater, to store a second vaporization substance for vaporization by the vapor that is heated by the heater.

32-33. (canceled)

34. The apparatus of claim 31, further comprising:
a channel in fluid communication with the atomizer and the second chamber,
wherein at least a portion of the heater is inside the channel.

35. The apparatus of claim 31, further comprising:
a channel in fluid communication with the atomizer and the second chamber,
a regulator to control movement of the second vaporization substance from the second chamber to the channel,
a user input device to control the regulator to permit or inhibit the movement of the second vaporization substance from the second chamber to the channel,
wherein the user input device further controls power to the heater.

36-53. (canceled)

54. The apparatus of claim 31, wherein the heater comprises at least one of a coil heater, a fan heater, a ceramic heater, and a quartz heater.

55-59. (canceled)

60. The apparatus of claim 31, wherein the second chamber is one of a plurality of chambers in fluid communication with the heater, to store respective vaporization substances for vaporization by the vapor that is heated by the heater.

61. A method comprising:
providing a first chamber to store a first vaporization substance;
providing an atomizer, in fluid communication with the first chamber, to generate vapor from the first vaporization substance by heating the first vaporization substance;
providing a channel in fluid communication with the atomizer;
providing a second chamber to store a second vaporization substance;
providing a feeder, in fluid communication with the channel and the second chamber, to feed the second vaporization substance from the second chamber to the channel.

62. The method of claim 61, further comprising:
arranging the feeder downstream from the atomizer;
wherein the second vaporization substance is vaporized by heat from the vapor.

63. The method of claim 61, wherein providing the feeder comprises providing an unheated atomizer.

64. (canceled)

65. The method of claim 61, further comprising:
arranging at least a portion of the feeder inside the channel.

66. The method of claim 61, further comprising:
arranging at least a portion of the feeder inside a separate channel that is in fluid communication with the channel.

67. The method of claim 61, wherein providing the feeder comprises providing a regulator to control movement of the second vaporization substance from the second chamber to the channel,
wherein providing the regulator comprises any one or more of: providing a wick, providing a valve, providing a pump, providing a spray nozzle, providing a mechanical feed structure, and providing a screw conveyor.

68. The method of claim 61, wherein providing the feeder comprises providing a regulator to control movement of the second vaporization substance from the second chamber to the channel, the method further comprising:
providing a user input device to control the regulator to permit or inhibit the movement of the second vaporization substance from the second chamber to the channel.

69-74. (canceled)

75. The method of claim 61, wherein providing the feeder comprises providing a holder to hold the second vaporization substance in the channel,
wherein providing the holder comprises any one or more of: providing a wick, providing an absorbent material, and providing an air permeable material.

76. The method of claim 61, wherein providing the feeder comprises providing a holder to hold the second vaporization substance in the channel, the method further comprising:
providing a user input device to control exposure of the holder to the channel.

77-87. (canceled)

88. The method of claim 61, further comprising:
providing a further chamber and a further feeder in fluid communication with the further chamber and the channel.

89. The method of claim 61, further comprising:
providing a heater, in fluid communication with the atomizer, to heat the vapor from the atomizer to vaporize the second vaporization substance.

90-116. (canceled)

117. A method of use of the apparatus of claim 1, the method comprising:
initiating vaporization of the first vaporization substance to produce a first vapor;
initiating feeding of the second vaporization substance into the channel to produce a second vapor; and
inhaling the first vapor and the second vapor.

118. A method of use of the apparatus of claim 31, the method comprising:
initiating vaporization of the first vaporization substance to produce a first vapor;
initiating heating of the first vapor;
initiating vaporization of the second vaporization substance by the first vapor that is heated by the heater, to produce a second vapor; and
inhaling the first vapor and the second vapor.
